(19) United States
(12) Patent Application Publication
(10) Pub. No.: US 2024/0309361 A1
(43) Pub. Date: Sep. 19, 2024

(54) METHODS

(71) Applicant: bluebird bio, Inc., Somerville, MA (US)

(72) Inventors: Ilya Shestopalov, Billerica, MA (US); Gregory Lawrence Saia, Medford, MA (US)

(21) Appl. No.: 18/576,073

(22) PCT Filed: Jun. 30, 2022
(86) PCT No.: PCT/US2022/035883

§ 371 Date: Jan. 2, 2024

Related U.S. Application Data
(60) Provisional application No. 63/217,576, filed on Jul. 1, 2021.

(51) Int. Cl. C12N 15/10

(52) U.S. Cl. CPC C12N 15/1082

(57) ABSTRACT

The present disclosure provides improved methods for measuring hemoglobin A (HbA) formation. More particularly, the disclosure provides methods for assessing potency of a viral vector encoding β-globin.

### RELATED APPLICATION

This application claims priority to, and the benefit of, co-pending U.S. Provisional Application No. 63/217,576, filed Jul. 1, 2021. The disclosure of said provisional application is hereby incorporated by reference in its entirety.

### BACKGROUND

### Technical Field

The present disclosure relates to improved methods for measuring hemoglobin A formation. More particularly, the disclosure provides improved methods for assessing potency of a vector (e.g., viral vector) encoding β-globin.

### Description of the Related Art

With the advent of gene therapy concepts in the 1960s and early 1970s, gene therapies have only recently made their debut in the clinic, and only a handful of gene therapies have been approved by the FDA. Gene therapy techniques include both viral vector and non-viral vector methods for transferring nucleic acids to a target cell.

Currently, there are several gene therapies in development for the treatment of various hemoglobinopathies, including thalassemia and sickle cell disease. Common to these diseases is a lack of functional Hemoglobin A (HbA). Although the gene therapy techniques being pursued vary in their mechanism of action, the end result is the same, to increase expression of functional HbA or HbF (Fetal Hemoglobin).

Accordingly, whether viral or non-viral vectors are used in a gene therapy to treat a hemoglobinopathy, both researchers and regulators require methods to assess the effectiveness/potency of the vector to transfer the desired nucleic acid (e.g., a nucleic acid encoding a therapeutic β-globin) and increase functional HbA and/or HbF expression. Current methods suffer from variable results, limited accuracy, poor specificity, and/or inability to compare across experiments and/or batches of vector. Accordingly, there remains a need for improved methods for detecting functional HbA and assessing vector potency.

### BRIEF SUMMARY

The present disclosure generally relates, in part, to methods for assessing functional hemoglobin A (HbA) formation. In some embodiments, the methods for assessing potency of a viral vector encoding a globin gene (e.g., a β-globin) are provided.

In one aspect, a method for assessing hemoglobin A (HbA) formation in cells is provided, comprising: modifying a population of cells to express a globin (e.g., β-globin); lysing the cells under non-denaturing conditions, thus forming cell lysates; analyzing the cell lysates with ion exchange (IEX) chromatography, and calculating HbA expression. In particular embodiments, the analyzing comprises passing the cell lysates through an IEX chromatographic column, and detecting heme groups associated with HbF and/or HbA hemoglobin multimers at 418 nm. In some embodiments, the HbA expression is calculated relative to a reference standard.

In various embodiments, the modifying comprises introducing a vector encoding a globin (e.g., β-globin) gene into the population of cells. In some embodiments, the vector is a viral vector or a non-viral vector. In some embodiments, the vector is introduced by transfection, transduction, or electroporation.

In various embodiments, the modifying comprises introducing into the population of cells: an endonuclease or polynucleotide encoding an endonuclease; and a donor repair template encoding a globin (e.g., β-globin). In some embodiments, the endonuclease is selected from the group consisting of: a homing endonuclease, or functional variant thereof; a megaTAL, or functional variant thereof; a CRISPR-associated nuclease, or functional variant thereof; a zinc-finger nuclease, or functional variant thereof; and transcription activator-like effector nuclease (TALEN), or functional variant thereof. In some embodiments, the endonuclease or polynucleotide encoding an endonuclease is introduced by transfection, transduction, or electroporation. In some embodiments, the donor repair template is introduced by transfection, transduction, or electroporation.

In various embodiments, the method further comprises culturing the cells for about 24 to about 96 hours post-modifying.

In another aspect a method for assessing potency of a viral vector encoding a globin gene (e.g. a β-globin) is provided, comprising: transducing a population of cells that do not express hemoglobin A (HbA) with a vector encoding a globin gene (e.g., β-globin); lysing the cells under non-denaturing conditions, thus forming cell lysates; analyzing the cell lysates with ion exchange (IEX) chromatography; and calculating HbA expression relative to HbA expression in a cell introduced with a reference standard vector. In particular embodiments, the analyzing comprises passing the cell lysates through an IEX chromatographic column, and detecting heme groups associated with HbF and/or HbA hemoglobin multimers at 418 nm.

In various embodiments, the potency is a relative potency. In some embodiments, the potency is relative to a reference standard.

In various embodiments, the method further comprises culturing the population of cells for 24 to 96 hours post-transduction.

In various embodiments, the population of cells do not endogenously express HbA. In some embodiments, the population of cells have been genetically edited to not express HbA. In some embodiments, the population of cells express fetal hemoglobin (HbF). In some embodiments, the population of cells are a myelogenous leukemia cell line. In particular embodiments, the population of cells are K562 cells.

In various embodiments, cells are plated at a cell density of about 0.5×10⁶ cells/ml, about 1.0×10⁶ cells/ml, about 1.5×10⁶ cells/ml, about 2.0×10⁶ cells/ml, about 2.5×10⁶ cells/ml, or about 3.0×10⁶ cells/ml prior to modification or transduction. In some embodiments, the cells are plated at a cell density of about 0.5×10⁶ cells/ml to about 3.0×10⁶ cells/ml prior to modification or transduction. In some embodiments, the cells are plated at a cell density of about 0.5×10⁶ cells/ml to about 2.5×10⁶ cells/ml prior to modification or transduction. In some embodiments, the cells are plated at a cell density of about 0.5×10⁶ cells/ml to about 2.0×10⁶ cells/ml prior to modification or transduction. In some embodiments, the cells are plated at a cell density of about 0.5×10⁶ cells/ml to about 1.5×10⁶ cells/ml prior to modification or transduction. In some embodiments, the cells are plated at a cell density of about 0.6×10⁶ cells/ml to about 1.4×10⁶ cells/ml prior to modification or transduction. In some embodiments, the cells are plated at a cell density of about 0.7×10⁶ cells/ml to about 1.3×10⁶ cells/ml prior to modification or transduction. In some embodiments, the cells are plated at a cell density of about 0.8×10⁶ cells/ml to about 1.2×10⁶ cells/ml prior to modification or transduction. In some embodiments, the cells are plated at a cell density of about 0.9×10⁶ cells/ml to about 1.1×10⁶ cells/ml prior to modification or transduction. In some embodiments, the cells are plated at a cell density of about 1.0×10⁶ cells/ml prior to modification or transduction.

In various embodiments, the cells are plated in tissue culture flasks. In various embodiments, the cells are plated in a 12-well plate. In various embodiments, the cells are plated in a 24-well plate.

In various embodiments, cells are plated in a total volume of about 1 ml. In various embodiments, the cells are plated in a total volume of about 2 ml.

In various embodiments, the cells are transduced in the presence of polybrene. In some embodiments, the cells are transduced in the presence of about 2 μg/ml to about 8 μg/ml polybrene. In some embodiments, the cells are transduced in the presence of about 8 μg/ml polybrene.

In various embodiments, the cells are cultured for about 48 to about 96 hours post-modification or -transduction. In some embodiments, the cells are cultured for about 60 to about 84 hours post-modification or -transduction. In some embodiments, the cells are cultured for about 48, about 60, about 72, about 84, or about 96 hours post-modification or -transduction. In some embodiments, the cells are cultured for about 72 hours post-modification or -transduction. In some embodiments, the cells are cultured for about 72±2 hours post-modification or -transduction.

In various embodiments, the cells are frozen after lysis and prior to analyzing the cell lysates with ion exchange (IEX) chromatography.

In various embodiments, the HbF comprises a and y globin chain dimers or tetramers.

In various embodiments, the HbA comprises a and B globin chain dimers or tetramers. In some embodiments, the β-globin is a human β-globin. In some embodiments, the β-globin is β^{A-T87Q} globin, a β^{A-G16D/E22A/T87Q}-globin, or a β^{A-T87Q/K95E/K120E}-globin.

In various embodiments, the vector is a lentiviral vector. In some embodiments, the vector is an AnkT9W vector, a T9Ank2W vector, a TNS9 vector, a lentiglobin HPV569 vector, a lentiglobin BB305 vector, a BG-1 vector, a BGM-1 vector, a d432βAγ vector, a mLARβΔγV5 vector, a GLOBE vector, a G-GLOBE vector, a βAS3-FB vector, a V5 vector, a V5m3 vector, a V5m3-400 vector, and a G9 vector, or a derivative thereof. In particular embodiments, the vector is bb305.

In various embodiments, the transducing comprises transduction of vector at a multiplicity of infection (MOI) of about 5 to about 40, about 5 to about 30, about 10 to about 40, or about 10 to about 30. In some embodiments, the transducing comprises transduction of vector at a multiplicity of infection (MOI) of 5-40, 5-30, 10-40, or 10-30. In some embodiments, the transducing comprises transduction of vector at a multiplicity of infection (MOI) of about 5, about 10, about 15, about 20, about 25, about 30, about 35, and/or about 40. In some embodiments, the transducing comprises transduction with vector at a multiplicity of infection (MOI) of about 20. In some embodiments, the transducing comprises transduction with vector at one or more MOIs in different wells or plates. In some embodiments, the transducing comprises transduction with vector at one or more MOIs in different wells or plates in duplicate. In some embodiments, the transducing comprises transduction with vector at one or more MOIs in different wells or plates in triplicate. In some embodiments, the transducing comprises transduction with vector at MOIs of 10, 15, 20, 25, and 30.

In various embodiments, the IEX chromatography is IEX HPLC. In some embodiments, the IEX chromatography is IEX UPLC™. In some embodiments, the IEX chromatography is IEX UHLPC.

In various embodiments, the IEX chromatography comprises liquid-based first and second mobile phases. In some embodiments, the column comprises a solid phase comprising aspartic acid chains covalently linked to a substrate. In some embodiments, the column comprises a solid phase comprising sulfonic acid ligands covalently lined to a substrate. In some embodiments, the substrate is a silica substrate. In some embodiments, the substrate is a polymer.

In various embodiments, the chromatography comprises a tunable ultraviolet (TUV) detector.

In various embodiments, the chromatography comprises a photodiode array ultraviolet (PDA UV) detector.

In various embodiments, the chromatography separates HbF multimers from HbA multimers. In some embodiments, chromatographic identification of HbA and HbF is made based on the matched retention time of the analyte peaks relative to a hemoglobin standard. In some embodiments, the standard is AFSC.

In various embodiments, the calculating comprises determining an HbA peak and measuring the area under the curve (AUC). In some embodiments, the calculating comprises determining an HbF peak and measuring the area under the curve (AUC). In some embodiments, the calculating comprises determining HbA expression as a percentage of HbA relative to the sum of HbA and HbF. In some embodiments, the calculating further comprises fitting a log-dose response curve to the calculated HbA expression.

In various embodiments, the calculating further comprises fitting a linear log-dose response curve to a reference standard and the vector. In some embodiments, the log-dose is a log₁₀ dose.

In various embodiments, the fitting comprises a parallel line approach to determine a relative potency. In some embodiments, the relative potency is determined by the formula:

$$Relative potency = antilog ( \frac{Test intercept - Reference Intercept}{Common Slope} )$$

In various embodiments, the fitting comprises an interpolation approach. In some embodiments, the interpolation approach comprises a linear fit applied to the reference standard log-dose response and the % HbA responses of the vector are used to interpolate MOI from the reference curve fit.

In various embodiments, the method is an in vitro method.

In various embodiments, the method is an ex vivo method.

### BRIEF DESCRIPTION OF SEVERAL VIEWS OF THE DRAWINGS

FIG. 1A shows a representative chromatogram demonstrating a lentiviral vector derived Hemoglobin A (HbA) peak and a Hemoglobin F (HbF) peak.

FIG. 1B shows percent HbA (% HbA) based on the HbA peak area relative to the sum of HbA and HbF peak areas at different multiplicities of infection (MOIs), with and without polybrene.

FIG. 1C shows % HbA over time as a function of starting cell density.

FIG. 2A shows % HbA plotted against the log₁₀ MOI at various harvest times for lentiviral vector lot 2.

FIG. 2B shows % HbA plotted against the log₁₀ MOI at various harvest times for lentiviral vector lot 3.

FIG. 3 shows representative chromatograms of cell lysates derived from lentiviral vector (LentiGlobin BB305 LVV), GFP, and mock, transfected cells.

FIG. 4A shows % HbA at different MOIs and under varying culture conditions.

FIG. 4B shows HbA area under the curve (AUC) at different MOIs and under varying culture conditions.

FIG. 4C shows HbF area under the curve (AUC) at different MOIs and under varying culture conditions.

FIG. 5A shows % HbA calculations from cells transduced with lentiviral vector encoding β-globin.

FIG. 5B shows HbA and HbF AUC from cells transduced with lentiviral vector encoding β-globin.

FIG. 5C shows HbF AUC of mock-transduced samples.

FIG. 6A shows % HbA calculations from cells transduced with lentiviral vector from adherent (reference lot 19) and suspension (lot 003) production cell lines.

FIG. 6B shows % HbA calculations from cells transduced with lentiviral vector from adherent (reference lot 19) and suspension (lot 004) production cell lines.

FIG. 7A shows expected vs observed peak AUC for HbA and HbF peaks.

FIG. 7B shows expected vs observed % HbA

FIG. 8 shows representative chromatograms from cell lysates derived from lentiviral vector (LentiGlobin BB305 LVV) and mock transfected cells, compared to blank and AFSC hemoglobin standard conditions.

### DETAILED DESCRIPTION

### A. Overview

The present disclosure generally relates to, in part, improved methods of detecting hemoglobin A expression/formation. More particularly, the disclosure relates to improved methods for assessing potency of a vector (e.g., viral vector) encoding β-globin. Specifically, the inventors have discovered an improved hemoglobin detection and vector potency assays that are both highly accurate and specific for the detection of HbA and HbF, while also having the ability to compare results across experiments and different batches/lots of vector. Moreover, the assay can be completed in a relatively short timeframe, e.g., cell preparation in 7 days or less, and chromatography in 1 day. Accordingly, the problems of limited accuracy, poor specificity, speed, and/or lack of comparability are solved by the improved methods, as described further herein.

In one aspect, a method for assessing hemoglobin A expression is provided, comprising: modifying a population of cells to express a β-globin gene; lysing the cells under non-denaturing conditions, thus forming cell lysates; analyzing the cell lysates with ion exchange (IEX) chromatography, and calculating HbA expression. In various embodiments, the modifying comprises (i) introducing into the population of cells a vector encoding a β-globin gene into the populations of cells, or (ii) introducing into the population of cells an endonuclease or polynucleotide encoding an endonuclease (e.g., a homing endonuclease, megaTAL, CRISPR-associated nuclease, zinc-finger nuclease, or TALEN), and a donor repair template encoding a β-globin gene.

In another aspect, a method for assessing potency of a viral vector encoding a β-globin gene is provided, comprising: transducing a population of cells that do not express HbA with a vector encoding a β-globin gene; lysing the cells under non-denaturing conditions, thus forming cell lysates; analyzing the cell lysates with ion exchange (IEX) chromatography, and calculating HbA expression relative to HbA expression in a cell introduced with a reference standard vector.

In various embodiments, the methods described herein further comprise culturing the cells for about 24 to 96 hours post-modifying or -transducing.

In various embodiments, the IEX chromatography comprises: passing the cell lysates through an IEX chromatographic column; and detecting heme groups associated with HbF and/or HbA hemoglobin multimers at 418 nm. In some embodiments, the IEX chromatography is HPLC, UPLC, or UHPLC.

In various embodiments, the population of cells do not endogenously express HbA or have been genetically edited to not express HbA. In various embodiments, the cells express fetal hemoglobin (HbF). In some embodiments, the cells are a myelogenous leukemia cell line (e.g., K562 cells). In some embodiments, the cells are plated at a cell density of about 0.5×10⁶ cells/ml to about 3.0×10⁶ cells/ml prior to modification or transduction.

In various embodiments, the transducing comprises transduction of vector at a multiplicity of infection (MOI) of about 5 to about 40, about 5 to about 30, about 10 to about 40, or about 10 to about 30. In some embodiments, the transducing comprises transduction with vector at one or more MOIs in different wells or plates.

In various embodiments, the potency is a relative potency. In some embodiments, the calculating comprises determining an HbA peak and/or HbF and measuring the area under the curve (AUC). In some embodiments, the calculating comprises determining HbA expression as a percentage of HbA relative to the sum of HbA and HbF.

In various embodiments, the calculating further comprises fitting log-dose (e.g., log₁₀) response curves to the calculated HbA expression. In some embodiments, the fit is a linear fit. In some embodiments, the fitting comprises a parallel line approach or an interpolation approach. In some embodiments, the interpolation approach comprises a linear fit applied to the reference standard log-dose response and the % HbA responses of the vector are used to interpolate MOI from the reference curve fit.

In particular embodiments, the chromatographic identification of HbA and HbF is made based on the matched retention time of the analyte peaks relative to a hemoglobin standard (e.g., AFSC).

Techniques for recombinant (i.e., engineered) DNA, peptide and oligonucleotide synthesis, immunoassays, tissue culture, transformation (e.g., electroporation, lipofection), enzymatic reactions, purification and related techniques and procedures may be generally performed as described in various general and more specific references in microbiology, molecular biology, biochemistry, molecular genetics, cell biology, virology and immunology as cited and discussed throughout the present specification. See, e.g., Sambrook et al., Molecular Cloning: A Laboratory Manual, 3d ed., Cold Spring Harbor Laboratory Press, Cold Spring Harbor, N.Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, NY); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid The Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3rd Edition, 2010 Humana Press); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir and CC Blackwell, eds., 1986); Roitt, Essential Immunology, 6th Edition, (Blackwell Scientific Publications, Oxford, 1988); Current Protocols in Immunology (Q. E. Coligan, A. M. Kruisbeek, D. H. Margulies, E. M. Shevach and W. Strober, eds., 1991); Annual Review of Immunology; as well as monographs in journals such as Advances in Immunology.

### B. Definitions

Prior to setting forth this disclosure in more detail, it may be helpful to an understanding thereof to provide definitions of certain terms to be used herein.

Unless defined otherwise, all technical and scientific terms used herein have the same meaning as commonly understood by those of ordinary skill in the art to which the invention belongs. Although any methods and materials similar or equivalent to those described herein can be used in the practice or testing of particular embodiments, preferred embodiments of compositions, methods and materials are described herein. For the purposes of the present disclosure, the following terms are defined below.

The articles “a,” “an,” and “the” are used herein to refer to one or to more than one (i.e., to at least one, or to one or more) of the grammatical object of the article. By way of example, “an element” means one element or one or more elements.

The use of the alternative (e.g., “or”) should be understood to mean either one, both, or any combination thereof of the alternatives.

The term “and/or” should be understood to mean either one, or both of the alternatives.

As used herein, the term “about” or “approximately” refers to a quantity, level, value, number, frequency, percentage, dimension, size, amount, weight or length that varies by no more than 15%, 10%, 9%, 8%, 7%, 6%, 5%, 4%, 3%, 2% or 1% to a reference quantity, level, value, number, frequency, percentage, dimension, size, amount, weight or length. In one embodiment, the term “about” or “approximately” refers a range of quantity, level, value, number, frequency, percentage, dimension, size, amount, weight or length ±15%, ±10%, ±9%, ±8%, ±7%, ±6%, ±5%, ±4%, ±3%, ±2%, or ±1% about a reference quantity, level, value, number, frequency, percentage, dimension, size, amount, weight or length.

In one embodiment, a range, e.g., 1 to 5, about 1 to 5, or about 1 to about 5, refers to each numerical value encompassed by the range. For example, in one non-limiting and merely illustrative embodiment, the range “1 to 5” is equivalent to the expression 1, 2, 3, 4, 5; or 1.0, 1.5, 2.0, 2.5, 3.0, 3.5, 4.0, 4.5, or 5.0; or 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0.

As used herein, the term “substantially” refers to a quantity, level, value, number, frequency, percentage, dimension, size, amount, weight or length that is 80%, 85%, 90%, 91%, 92%, 93%, 94%, 95%, 96%, 97%, 98%, 99% or higher compared to a reference quantity, level, value, number, frequency, percentage, dimension, size, amount, weight or length. In one embodiment, “substantially the same” refers to a quantity, level, value, number, frequency, percentage, dimension, size, amount, weight or length that produces an effect, e.g., a physiological effect, that is approximately the same as a reference quantity, level, value, number, frequency, percentage, dimension, size, amount, weight or length.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are present that materially affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment. Thus, the appearances of the foregoing phrases in various places throughout this specification are not necessarily all referring to the same embodiment. Furthermore, the particular features, structures, or characteristics may be combined in any suitable manner in one or more embodiments. It is also understood that the positive recitation of a feature in one embodiment, serves as a basis for excluding the feature in a particular embodiment.

The term “transfection” as used herein refers to the process of introducing naked DNA into cells by non-viral methods, e.g., by use of liquid nano particles.

The term “infection” as used herein refers to the process of introducing foreign DNA into cells using a viral vector.

The term “transduction” as used herein refers to the introduction of foreign DNA into a cell's genome using a viral vector.

The term “globin” as used herein refers to proteins or protein subunits that are capable of covalently or noncovalently binding a heme moiety, and can therefore transport or store oxygen. Subunits of vertebrate and invertebrate hemoglobins, vertebrate and invertebrate myoglobins or mutants thereof are included by the term globin. The term excludes hemocyanins. Examples of globins include α-globin or variants thereof, β-globin or variants thereof, a γ-globin or variants thereof, and δ-globin or variants thereof.

The term “hemoglobin” refers to a multimeric, iron-containing, oxygen-transport metalloprotein in the red blood cells (erythrocytes). Hemoglobin is typically a quaternary structure, i.e., it comprises four protein subunits (globin molecules), and each subunit/chain is tightly associated with a non-protein heme group. For example, Hemoglobin A (HbA) “multimers” comprises α- and β-globin chain dimers or tetramers. Hemoglobin F (HbF) “multimers” comprises α- and γ-globin chain dimers or tetramers.

An “exogenous” molecule is a molecule that is not normally present in a cell, but that is introduced into a cell by one or more genetic, biochemical or other methods. Exemplary exogenous molecules include, but are not limited to small organic molecules, protein, nucleic acid, carbohydrate, lipid, glycoprotein, lipoprotein, polysaccharide, any modified derivative of the above molecules, or any complex comprising one or more of the above molecules. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, biopolymer nanoparticle, calcium phosphate co-precipitation, DEAE-dextran-mediated transfer and viral vector-mediated transfer.

An “endogenous” molecule is one that is normally present in a particular cell at a particular developmental stage under particular environmental conditions. For example, an endogenous nucleic acid can comprise a chromosome, the genome of a mitochondrion, or other organelle, or a naturally-occurring episomal nucleic acid. Additional endogenous molecules can include proteins, for example, endogenous β-globin or γ-globin.

A “gene,” refers to a DNA region encoding a gene product, as well as all DNA regions which regulate the production of the gene product, whether or not such regulatory sequences are adjacent to coding and/or transcribed sequences. A gene includes, but is not limited to, promoter sequences, enhancers, silencers, insulators, boundary elements, terminators, polyadenylation sequences, post-transcription response elements, translational regulatory sequences such as ribosome binding sites and internal ribosome entry sites, replication origins, matrix attachment sites, and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA). Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

As used herein, the term “genome editing”, “gene edited”, or “genetically edited” refers to the substitution, deletion, and/or introduction of genetic material at a target site in the cell's genome, which restores, corrects, and/or modifies expression of a gene. Genome editing contemplated in particular embodiments comprises introducing one or more endonucleases or polynucleotides encoding an endonuclease into a cell to generate DNA lesions at a target site in the cell's genome, and to disrupt, reduce, or eliminate expression of a globin or globin multimer (e.g., β-globin and/or HbA). Genome editing contemplated in yet other embodiments, comprises introducing one or more endonucleases or polynucleotides encoding an endonuclease, and a donor repair template encoding a globin (β-globin) into a cell.

Additional definitions are set forth throughout this disclosure.

### C. Methods for Determining HBA Expression

There are many types and variants of hemoglobin, including, but not limited to, Hemoglobin A (HbA), Fetal Hemoglobin (HbF), Hemoglobin S (HbS), and Hemoglobin C (HbC). HbA, also known as adult hemoglobin, consists of α- and β-globin chain dimers or tetramers, and is the primary oxygen binding protein in the adult human. HbF, consists of α- and γ-globin chain dimers and tetramers, and is the primary oxygen binding protein in the human fetus. HbS and HbC are abnormal hemoglobin variants associated with sickle cell disease and sickle cell trait, respectively.

As discussed throughout this disclosure, the inventors have surprisingly discovered methods for assessing HbA expression and vector potency that demonstrates specificity, robustness, precision, and linearity across a range that is suitable for the determination of % HbA in cell pellet samples. The disclosure also relates to improved methods for assessing potency of a vector (e.g., viral vector) encoding a globin (e.g., β-globin).

In one aspect, a method for assessing hemoglobin A (HbA) formation in cells is provided. comprising: modifying a population of cells to express β-globin; lysing the cells under non-denaturing conditions, thus forming cell lysates; analyzing the cell lysates with ion exchange (IEX) chromatography, and calculating HbA expression.

In another aspect, a method for assessing potency of a viral vector encoding a β-globin gene is provided, comprising: transducing a population of cells that do not express HbA with a vector encoding a β-globin gene; lysing the cells under non-denaturing conditions, thus forming cell lysates; analyzing the cell lysates with ion exchange (IEX) chromatography, and calculating HbA expression relative to a reference standard vector. In particular embodiments, the potency is a relative potency.

In various embodiments, the methods describe herein comprise a step of introducing a vector into a cell. In various embodiments, the vector is a viral or non-viral vector. Vector types and methods for introducing vectors into cells (e.g., by transduction, transfection, lipofection, or electroporation) are known in the art and discussed further below. In brief, in certain embodiments, the vector comprises a polynucleotide encoding a globin. In some embodiments, the globin is a human β-globin, a human δ-globin, an anti-sickling globin, a human γ-globin, a human β^{A-T87Q}-globin, a human β^{A-G16D/E22A/T87Q}-globin, or a human β^{A-T87Q/K95F/K120E}-globin protein. In certain embodiments, the globin is a human β-globin protein. In certain embodiments, the globin is an anti-sickling globin protein. In certain embodiments, the globin is a human γ-globin protein. In certain embodiments, the globin is a human β^{A-T87Q}-globin protein. In certain aspects, the globin is a human β^{A-G16D/E22A/T87Q}-globin protein. In certain aspects, the globin is a human β^{A-T87Q/K95F/K120E}-globin protein. In certain embodiments, the β-globin is a human β-globin. In particular embodiments, the β-globin is β^{A-T87Q} globin.

In various embodiments, the vector is a lentiviral vector. In some embodiments, the lentiviral vector is an AnkT9W vector, a T9Ank2W vector, a TNS9 vector, a TNS9.3 vector, a TNS9.3.55 vector, a lentiglobin HPV569 vector, a lentiglobin BB305 vector, a BG-1 vector, a BGM-1 vector, a mLARβΔγV5 vector, a GLOBE vector, a G-GLOBE vector, a βAS3-FB vector, a V5 vector, a V5m3 vector, a V5m3-400 vector, a G9 vector, or a derivative thereof.

In some embodiments, the lentiviral vector is an AnkT9W vector or a derivative thereof. In some embodiments, the lentiviral vector is a T9Ank2W vector or a derivative thereof. In some embodiments, the lentiviral vector is a TNS9 vector or a derivative thereof. In some embodiments, the lentiviral vector is a TNS9.3 vector or a derivative thereof. In some embodiments, the lentiviral vector is a TNS9.3.55 vector or a derivative thereof. In some embodiments, the lentiviral vector is a lentiglobin HPV569 vector or a derivative thereof. In some embodiments, the lentiviral vector is a lentiglobin BB305 vector or a derivative thereof. In some embodiments, the lentiviral vector is a BG-1 vector or a derivative thereof. In some embodiments, the lentiviral vector is a BGM-1 vector or a derivative thereof. In some embodiments, the lentiviral vector is a mLARβΔγV5 vector, or a derivative thereof. In some embodiments, the lentiviral vector is a GLOBE vector or a derivative thereof. In some embodiments, the lentiviral vector is a G-GLOBE vector or a derivative thereof. In some embodiments, the lentiviral vector is a βAS3-FB vector or a derivative thereof. In some embodiments, the lentiviral vector is a V5 vector. In some embodiments, the lentiviral vector is a V5m3 vector, or a derivative thereof. In some embodiments, the lentiviral vector is a V5m3-400 vector, or a derivative thereof. In some embodiments, the lentiviral vector is a G9 vector, or a derivative thereof.

In particular embodiments, the lentiviral vector is bb305.

In various embodiments, the modifying comprises introducing into the population of cells: an endonuclease or polynucleotide encoding an endonuclease; and a donor repair template or vector encoding a globin gene (e.g., β-globin). In some embodiments, the endonuclease is a homing endonuclease (HE), also known as a meganuclease, or functional variant thereof. In some embodiments, the endonuclease is a megaTAL, or functional variant thereof. In some embodiments, the endonuclease is a CRISPR-associated (Cas) nuclease, or functional variant thereof. In some embodiments, the endonuclease is a transcription activator-like effector nuclease (TALEN), or functional variant thereof.

In various embodiments, the endonuclease or polynucleotide encoding an endonuclease is introduced by transfection, transduction, or electroporation.

In various embodiments, the donor repair template is introduced by transfection, transduction, or electroporation.

Cells useful for the methods described herein can be any cell that does not express HbA, or express low levels of HbA (e.g., at least 2-fold, 3-fold, 4-fold, 5-fold, 6-fold, 7-fold, 8-fold, 9-fold or 10-fold, less expression as compared to HbF expression). In some embodiments, the cell is a myelogenous leukemia cell line. In certain embodiments, a myelogenous leukemia cell line does not endogenously express HbA, or expresses low levels of HbA (e.g., at least 2-fold, 3-fold, 4-fold, 5-fold, 6-fold, 7-fold, 8-fold, 9-fold or 10-fold, less expression as compared to HbF expression).

HbA comprises α- and β-globin chain dimers or tetramers. Accordingly, in certain embodiments, the cells do not express β-globin. In some embodiments, the cells express α-globin. In particular embodiments, the cells express α-globin, and do not express β-globin.

HbF comprises α and γ globin chain dimers or tetramers. Accordingly, in various embodiments, the cells express HbF. In some embodiments, the cells endogenously express HbF. In some embodiments, the cells exogenously express HbF. Accordingly, in certain embodiments, the cell endogenously or exogenously express γ-globin. In particular embodiments, the cell endogenously or exogenously express α- and γ-globin. In even more particular embodiments, the cell endogenously or exogenously express α- and γ-globin chain dimers or tetramers. In a preferred embodiment, the cells are K562 cells.

In various embodiments, the cells are plated at a cell density of about 0.5×10⁶ cells/ml to about 3.0×10⁶ cells/ml prior to introduction of the vector (e.g., by transduction) or modification. In some embodiments, the cells are plated at a cell density of about 1.0×10⁶ cells/ml to about 3.0×10⁶ cells/ml prior to introduction of the vector or modification. In some embodiments, the cells are plated at a cell density of about 1.5×10⁶ cells/ml to about 3.0×10⁶ cells/ml prior to introduction of the vector or modification. In some embodiments, the cells are plated at a cell density of about 2.0×10⁶ cells/ml to about 3.0×10⁶ cells/ml prior to introduction of the vector or modification. In some embodiments, the cells are plated at a cell density of about 2.5×10⁶ cells/ml to about 3.0×10⁶ cells/ml prior to introduction of the vector or modification. In some embodiments, the cells are plated at a cell density of about 0.5×10⁶ cells/ml to about 2.5×10⁶ cells/ml prior to introduction of the vector or modification. In some embodiments, the cells are plated at a cell density of about 0.5×10⁶ cells/ml to about 2.0×10⁶ cells/ml prior to introduction of the vector or modification. In some embodiments, the cells are plated at a cell density of about 0.5×10⁶ cells/ml to about 1.5×10⁶ cells/ml prior to introduction of the vector or modification. In some embodiments, the cells are plated at a cell density of about 0.5×10⁶ cells/ml to about 1.0×10⁶ cells/ml prior to introduction of the vector or modification.

In various embodiments, the cells are plated at a cell density of about 0.6×10⁶ cells/ml to about 1.4×10⁶ cells/ml prior to introduction of the vector or modification. In some embodiments, the cells are plated at a cell density of about 0.7×10⁶ cells/ml to about 1.3×10⁶ cells/ml prior to introduction of the vector or modification. In some embodiments, the cells are plated at a cell density of about 0.8×10⁶ cells/ml to about 1.2×10⁶ cells/ml prior to introduction of the vector or modification. In some embodiments, the cells are plated at a cell density of about 0.9×10⁶ cells/ml to about 1.1×10⁶ cells/ml prior to introduction of the vector or modification.

In some embodiments, the cells are plated at a cell density of about 0.5×10⁶ cells/ml prior to introduction of the vector or modification. In some embodiments, the cells are plated at a cell density of about 0.6×10⁶ cells/ml prior to introduction of the vector or modification. In some embodiments, the cells are plated at a cell density of about 0.7×10⁶ cells/ml prior to introduction of the vector or modification. In some embodiments, the cells are plated at a cell density of about 0.8×10⁶ cells/ml prior to introduction of the vector or modification. In some embodiments, the cells are plated at a cell density of about 0.9×10⁶ cells/ml prior to introduction of the vector or modification. In some embodiments, the cells are plated at a cell density of about 1.0×10⁶ cells/ml prior to introduction of the vector or modification. In some embodiments, the cells are plated at a cell density of about 1.1×10⁶ cells/ml prior to introduction of the vector or modification. In some embodiments, the cells are plated at a cell density of about 1.2×10⁶ cells/ml prior to introduction of the vector or modification. In some embodiments, the cells are plated at a cell density of about 1.3×10⁶ cells/ml prior to introduction of the vector or modification. In some embodiments, the cells are plated at a cell density of about 1.4×10⁶ cells/ml prior to introduction of the vector or modification. In some embodiments, the cells are plated at a cell density of about 1.5×10⁶ cells/ml prior to introduction of the vector or modification. In some embodiments, the cells are plated at a cell density of about 1.6×10⁶ cells/ml prior to introduction of the vector or modification. In some embodiments, the cells are plated at a cell density of about 1.7×10⁶ cells/ml prior to introduction of the vector or modification. In some embodiments, the cells are plated at a cell density of about 1.8×10⁶ cells/ml prior to introduction of the vector or modification. In some embodiments, the cells are plated at a cell density of about 1.9×10⁶ cells/ml prior to introduction of the vector or modification. In some embodiments, the cells are plated at a cell density of about 2.0×10⁶ cells/ml prior to introduction of the vector or modification. In some embodiments, the cells are plated at a cell density of about 2.1×10⁶ cells/ml prior to introduction of the vector or modification. In some embodiments, the cells are plated at a cell density of about 2.2×10⁶ cells/ml prior to introduction of the vector or modification. In some embodiments, the cells are plated at a cell density of about 2.3×10⁶ cells/ml prior to introduction of the vector or modification. In some embodiments, the cells are plated at a cell density of about 2.4×10⁶ cells/ml prior to introduction of the vector or modification. In some embodiments, the cells are plated at a cell density of about 2.5×10⁶ cells/ml prior to introduction of the vector or modification. In some embodiments, the cells are plated at a cell density of about 2.6×10⁶ cells/ml prior to introduction of the vector or modification. In some embodiments, the cells are plated at a cell density of about 2.7×10⁶ cells/ml prior to introduction of the vector or modification. In some embodiments, the cells are plated at a cell density of about 2.8×10⁶ cells/ml prior to introduction of the vector or modification. In some embodiments, the cells are plated at a cell density of about 2.9×10⁶ cells/ml prior to introduction of the vector or modification. In some embodiments, the cells are plated at a cell density of about 3.0×10⁶ cells/ml prior to introduction of the vector or modification.

In various embodiments, the cells are plated in a 12-well plate, 24-well plate or tissue culture flasks. In some embodiments, the cells are plated in a 12-well plate. In some embodiments, the cells are plated in a 24-well plate. In some embodiments, the cells are plated in tissue vulture flasks.

In some embodiments, the cells are plated in a total volume of about 1 ml. In some embodiments, the cells are plated in a total volume of 1 ml. In some embodiments, the cells are plated in a total volume of about 2 ml. In some embodiments, the cells are plated in a total volume of 2 ml.

In various embodiments, the cells are transduced in the presence of polybrene. In some embodiments, the cells are transduced in the presence of about 2 μg/ml to about 8 μg/ml polybrene. In some embodiments, the cells are transduced in the presence of about 3 μg/ml to about 8 μg/ml polybrene. In some embodiments, the cells are transduced in the presence of about 4 μg/ml to about 8 μg/ml polybrene. In some embodiments, the cells are transduced in the presence of about 5 μg/ml to about 8 μg/ml polybrene. In some embodiments, the cells are transduced in the presence of about 6 μg/ml to about 8 μg/ml polybrene. In some embodiments, the cells are transduced in the presence of about 7 μg/ml to about 8 μg/ml polybrene.

In some embodiments, the cells are transduced in the presence of about 2 μg/ml polybrene. In some embodiments, the cells are transduced in the presence of about 3 μg/ml polybrene. In some embodiments, the cells are transduced in the presence of about 4 μg/ml polybrene. In some embodiments, the cells are transduced in the presence of about 5 μg/ml polybrene. In some embodiments, the cells are transduced in the presence of about 6 μg/ml polybrene. In some embodiments, the cells are transduced in the presence of about 7 μg/ml polybrene. In some embodiments, the cells are transduced in the presence of about 8 μg/ml polybrene.

In various embodiments, the introduction of a vector into the cells comprises transducing a viral vector at a multiplicity of infection (MOI) of about 5 to about 40, about 5 to about 30, about 10 to about 40, or about 10 to about 30. In some embodiments, the introduction of a vector into the cells comprises transducing a viral vector at a multiplicity of infection (MOI) of about 5 to about 40. In some embodiments, the introduction of a vector into the cells comprises transducing a viral vector at a multiplicity of infection (MOI) of about 5 to about 30. In some embodiments, the introduction of a vector into the cells comprises transducing a viral vector at a multiplicity of infection (MOI) of about 10 to about 40. In some embodiments, the introduction of a vector into the cells comprises transducing a viral vector at a multiplicity of infection (MOI) of about 10 to about 30.

In some embodiments, the introduction of a vector into the cells comprises transducing a viral vector at a multiplicity of infection (MOI) of 5-40. In some embodiments, the introduction of a vector into the cells comprises transducing a viral vector at a multiplicity of infection (MOI) of 5-30. In some embodiments, the introduction of a vector into the cells comprises transducing a viral vector at a multiplicity of infection (MOI) of 10-40. In some embodiments, the introduction of a vector into the cells comprises transducing a viral vector at a multiplicity of infection (MOI) of 10-30.

In various embodiments, the introduction of a vector into the cells comprises transducing a viral vector at a multiplicity of infection (MOI) of about 5, about 10, about 15, about 20, about 25, about 30, about 35, and/or about 40. In some embodiments, the introduction of a vector into the cells comprises transducing a viral vector at a multiplicity of infection (MOI) of about 5. In some embodiments, the introduction of a vector into the cells comprises transducing a viral vector at a multiplicity of infection (MOI) of about 10. In some embodiments, the introduction of a vector into the cells comprises transducing a viral vector at a multiplicity of infection (MOI) of about 15. In some embodiments, the introduction of a vector into the cells comprises transducing a viral vector at a multiplicity of infection (MOI) of about 20. In some embodiments, the introduction of a vector into the cells comprises transducing a viral vector at a multiplicity of infection (MOI) of about 25. In some embodiments, the introduction of a vector into the cells comprises transducing a viral vector at a multiplicity of infection (MOI) of about 30. In some embodiments, the introduction of a vector into the cells comprises transducing a viral vector at a multiplicity of infection (MOI) of about 35. In some embodiments, the introduction of a vector into the cells comprises transducing a viral vector at a multiplicity of infection (MOI) of about 40.

In various embodiments, the introduction of a vector into the cells comprises transducing a viral vector at one or more MOIs in different wells or plates. In some embodiments, the introduction of a vector into the cells comprises transducing a viral vector at one or more MOIs in different wells or plates in duplicate. In some embodiments, the introduction of a vector into the cells comprises transducing a viral vector at one or more MOIs in different wells or plates in triplicate.

In various embodiments, the transducing comprises transduction with vector at MOIs of about 10, about 15, about 20, about 25, and about 30. In some embodiments, the transducing comprises transduction with vector at MOIs of 10, 15, 20, 25, and 30. In some embodiments, the transducing comprises transduction with vector at MOIs of about 5, about 10, about 20, and about 30. In some embodiments, the transducing comprises transduction with vector at MOIs of 5, 10, 20, and 30. In some embodiments, the transducing comprises transduction with vector at MOIs of about 10, about 20, about 30, and about 40. In some embodiments, the transducing comprises transduction with vector at MOIs of 10, 20, 30, and 40. In some embodiments, the transducing comprises transduction with vector at MOIs of about 5, about 10, about 20, about 30, and about 40. In some embodiments, the transducing comprises transduction with vector at MOIs of 5, 10, 20, 30, and 40. In some embodiments, the transducing comprises transduction with vector at MOIs of about 5, about 10, about 20, and about 40. In some embodiments, the transducing comprises transduction with vector at MOIs of 5, 10, 20, and 40.

In various embodiments, the method described herein comprise a step of culturing cells post-modification or -transduction. In various embodiments, the cells are cultured for about 24 to about 96 hours post-modification or -transduction. In some embodiments, the cells are cultured for about 36 to about 96 hours post-modification or -transduction. In some embodiments, the cells are cultured for about 48 to about 96 hours post-modification or -transduction. In some embodiments, the cells are cultured for about 60 to about 96 hours post-modification or -transduction. In some embodiments, the cells are cultured for about 72 to about 96 hours post-modification or -transduction. In some embodiments, the cells are cultured for about 84 to about 96 hours post-modification or -transduction. In some embodiments, the cells are cultured for about 60 to about 84 hours post-modification or -transduction.

In some embodiments, the cells are cultured for about 24 hours post-modification or -transduction. In some embodiments, the cells are cultured for about 36 hours post-modification or -transduction. In some embodiments, the cells are cultured for about 48 hours post-modification or -transduction. In some embodiments, the cells are cultured for about 60 hours post-modification or -transduction. In some embodiments, the cells are cultured for about 72 hours post-modification or -transduction. In some embodiments, the cells are cultured for about 84 hours post-modification or -transduction. In some embodiments, the cells are cultured for about 96 hours post-modification or -transduction.

In various embodiments, the cells are cultured for about 72±2 hours post-modification or -transduction.

In various embodiments, the methods described herein comprise a step of lysing the cells after culturing. In some embodiments, the lysing is done under non-denaturing conditions. In some embodiments, the non-denaturing conditions comprise freezing the cells. In some embodiments, the lysing comprises freezing the cells, resuspending the frozen cell lysate in water, centrifuging the lysate, and collecting the lysate for analysis. In some embodiments, the cells are frozen after lysis and prior to analyzing the cell lysates with chromatography.

In various embodiments, the method described herein comprise a step of analyzing the cell lysates. In certain embodiments, the analyzing comprises a step of passing the cell lysates through a chromatographic column.

In particular embodiments, the chromatography is ion-exchange (IEX) chromatography. In some embodiments, the chromatography is HPLC, UHPLC, or UPLC™. In some embodiments, the chromatography is HPLC. In some embodiments, the chromatography is UHPLC. In some embodiments, the chromatography is UPLC. In various embodiments the IEX chromatography is IEX HPLC. In some embodiments, the IEX chromatography is IEX UHPLC. In some embodiments, the IEX chromatography is IEX ULPC. Methods and systems for conducting HPLC. UHPLC, and/or UPLC are known in the art and discussed further below.

In various embodiments, the chromatography (e.g., IEX chromatography) comprises liquid-based first and second mobile phases. In various embodiments, the chromatography (e.g., IEX chromatography) comprises a solid phase. In some embodiments, the solid phase comprises aspartic acid chains covalently linked to a substrate. In some embodiments, the solid phase comprises sulfonic acid ligands covalently linked to a substrate. In some embodiments, the substrate is a silica substrate. In some embodiments, the substrate is a polymer.

In certain embodiments, the analyzing comprises, detecting heme groups associated with HbF and/or HbA hemoglobin multimers. In various embodiments, the analyzing/chromatographic system comprises a detector. In some embodiments, the analyzing/chromatographic system comprises a detector, wherein HbF and HbA multimers are detected by measuring absorbance at 418 nm. In some embodiments, the analyzing/chromatographic system comprises an ultraviolet (UV) detector. In some embodiments, the analyzing/chromatographic system comprises a tunable ultraviolet (TUV) detector. In some embodiments, the analyzing/chromatographic system comprises a photodiode array ultraviolet (PDA UV) detector. In various embodiments, the chromatography separates HbF multimers/peaks from HbA multimers/peaks as measured by the detector.

In various embodiments, the methods describe herein comprise a calculating step. In some embodiments, the calculating comprises determining an HbA peak and measuring the area under the curve (AUC). In some embodiments, the calculating comprises determining an HbF peak and measuring the area under the curve (AUC). In some embodiments, the calculating comprises determining HbA expression as a percentage of HbA relative to the sum of HbA and HbF.

In various embodiments, the calculating further comprises fitting log-dose response curves to a reference standard and the vector. In various embodiments, the calculating further comprises fitting linear log-dose response curves to a reference standard and the vector. In particular embodiments, the log-dose response curve is a log₁₀ dose response curve.

In various embodiments, the fitting comprises a parallel line approach to determine a relative potency. In some embodiments, the relative potency is determined by the formula:

$$Relative potency = antilog ( \frac{Test intercept - Reference Intercept}{Common Slope} )$$

In various embodiments, the fitting comprises an interpolation approach. In some embodiments, the interpolation approach comprises a linear fit applied to the reference standard log-dose response and the % HbA responses of the vector are used to interpolate MOI from the reference curve fit.

In some embodiments, the chromatographic identification of HbA and HbF is made based on the matched retention time of the analyte peaks relative to a hemoglobin standard. In some embodiments, the standard is AFSC.

### D. Vectors and Transduction

In certain embodiments, a one or more polynucleotides encoding a globin gene (e.g., a therapeutic β-globin) are introduced into a cell by viral or non-viral vectors/methods. In some embodiments, polynucleotides encoding a globin gene and/or endonuclease may be introduced into a cell by viral or non-viral vectors/methods. In particular embodiments, delivery of one or more polynucleotides encoding a globin gene and/or endonuclease may be provided by the same method or by different methods, and/or by the same vector or by different vectors. In certain embodiments, the cell does not express HbA (e.g., K562 cells) or is a cell genetically edited to not express HbA.

The term “vector” is used herein to refer to a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. In particular embodiments, the vector is a viral vector or a non-viral vector.

As will be evident to one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). The term “viral vector” may refer either to a virus or viral particle capable of transferring a nucleic acid into a cell or to the transferred nucleic acid itself. Viral vectors and transfer plasmids contain structural and/or functional genetic elements that are primarily derived from a virus.

In particular embodiments, non-viral vectors are used to deliver one or more polynucleotides contemplated herein to a cell that does not endogenously express HbA. In one embodiment, the vector is an in vitro synthesized or synthetically prepared mRNA or cDNA encoding a globin gene and/or endonuclease.

In some embodiments, the polynucleotide or vector introduced to the cells comprises a polynucleotide encoding a globin. In some embodiments, the globin is a human β-globin, a human δ-globin, an anti-sickling globin, a human γ-globin, a human β^{A-T87Q}-globin, a human β^{A-G16D/E22A/T87Q}-globin, or a human β^{A-T87Q/K95E/K120E}-globin protein. In some embodiments, the globin is a human β-globin protein. In some embodiments, the globin is a human δ-globin protein. In some embodiments, the globin is an anti-sickling globin protein. In some embodiments, the globin is a human γ-globin protein. In some embodiments, the globin is a human β^{A-T87Q}-globin protein. In some embodiments, the globin is a human β^{A-G16D/E22A/T87Q}-globin protein. In some embodiments, the globin is a human β^{A-T87Q/K95E/K120E}-globin protein.

Illustrative examples of non-viral vectors include, but are not limited to mRNA, plasmids (e.g., DNA plasmids or RNA plasmids), transposons, cosmids, and bacterial artificial chromosomes.

Illustrative methods of non-viral delivery of polynucleotides or vectors contemplated in particular embodiments include, but are not limited to: electroporation, sonoporation, lipofection, microinjection, biolistics, virosomes, liposomes, calcium phosphate, immunoliposomes, nanoparticles, polycation or lipid:nucleic acid conjugates, naked DNA, artificial virions, DEAE-dextran-mediated transfer, gene gun, and heat-shock. In particular embodiments, the polynucleotide or vector is introduced by transfection, transduction, or electroporation.

Illustrative examples of polynucleotide and/or vector delivery systems suitable for use in particular embodiments contemplated in particular embodiments include, but are not limited to those provided by Amaxa Biosystems, Maxcyte, Inc., BTX Molecular Delivery Systems, and Copernicus Therapeutics Inc. Lipofection reagents are sold commercially (e.g., Transfectam™ and Lipofectin™). Cationic and neutral lipids that are suitable for efficient receptor-recognition lipofection of polynucleotides have been described in the literature. See e.g., Liu et al. (2003) Gene Therapy. 10:180-187; and Balazs et al. (2011) Journal of Drug Delivery. 2011:1-12. Antibody-targeted, bacterially derived, non-living nanocell-based delivery is also contemplated in particular embodiments.

Illustrative examples of viral vector systems suitable for use in particular embodiments contemplated herein include but are not limited to adeno-associated virus (AAV), retrovirus, herpes simplex virus, adenovirus, and vaccinia virus vectors.

In various embodiments, one or more polynucleotides or vectors encoding a β-globin are introduced into a cell, e.g., K562 cell, by transducing the cell with a recombinant adeno-associated virus (rAAV), comprising the one or more polynucleotides.

AAV is a small (˜26 nm) replication-defective, primarily episomal, non-enveloped virus. AAV can infect both dividing and non-dividing cells and may incorporate its genome into that of the host cell. Recombinant AAV (rAAV) are typically composed of, at a minimum, a transgene and its regulatory sequences, and 5′ and 3′ AAV inverted terminal repeats (ITRs). The ITR sequences are about 145 bp in length. In particular embodiments, the rAAV comprises ITRs and capsid sequences isolated from AAV1, AAV2, AAV3, AAV4, AAV5, AAV6, AAV7, AAV8, AAV9, or AAV10.

In some embodiments, a chimeric rAAV is used the ITR sequences are isolated from one AAV serotype and the capsid sequences are isolated from a different AAV serotype. For example, a rAAV with ITR sequences derived from AAV2 and capsid sequences derived from AAV6 is referred to as AAV2/AAV6. In particular embodiments, the rAAV vector may comprise ITRs from AAV2, and capsid proteins from any one of AAV1, AAV2, AAV3, AAV4, AAV5, AAV6, AAV7, AAV8, AAV9, or AAV10. In a preferred embodiment, the rAAV comprises ITR sequences derived from AAV2 and capsid sequences derived from AAV6. In a preferred embodiment, the rAAV comprises ITR sequences derived from AAV2 and capsid sequences derived from AAV2.

In some embodiments, engineering and selection methods can be applied to AAV capsids to make them more likely to transduce cells of interest.

Construction of rAAV vectors, production, and purification thereof have been disclosed, e.g., in U.S. Pat. Nos. 9,169,494; 9,169,492; 9,012,224; 8,889,641; 8,809,058; and 8,784,799, each of which is incorporated by reference herein, in its entirety.

In various embodiments, one or more polynucleotides or vectors encoding a β-globin are introduced into a cell (e.g., K562 cell), by transducing the cell with a retrovirus, e.g., lentivirus, comprising the one or more polynucleotides.

As used herein, the term “retrovirus” refers to an RNA virus that reverse transcribes its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

The term “lentiviral vector” refers to a retroviral vector or plasmid containing structural and functional genetic elements, or portions thereof, including LTRs that are primarily derived from a lentivirus. The terms “lentiviral vector” and “lentiviral expression vector” may be used to refer to lentiviral transfer plasmids and/or infectious lentiviral particles in particular embodiments. Where reference is made herein to elements such as cloning sites, promoters, regulatory elements, heterologous nucleic acids, etc., it is to be understood that the sequences of these elements are present in RNA form in the lentiviral particles contemplated herein and are present in DNA form in the DNA plasmids contemplated herein.

As used herein, the term “lentivirus” refers to a group (or genus) of complex retroviruses. Illustrative lentiviruses include, but are not limited to: HIV (human immunodeficiency virus; including HIV type 1, and HIV 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In one embodiment, HIV based vector backbones (i.e., HIV cis-acting sequence elements) are preferred.

In various embodiments, a lentiviral vector contemplated herein comprises one or more LTRs, and one or more, or all, of the following accessory elements: a cPPT/FLAP, a Psi (Ψ) packaging signal, an export element, poly (A) sequences, and may optionally comprise a WPRE or HPRE, an insulator element, a selectable marker, and a cell suicide gene, as discussed elsewhere herein.

In particular embodiments, lentiviral vectors contemplated herein may be integrative or non-integrating or integration defective lentivirus. As used herein, the term “integration defective lentivirus” or “IDLV” refers to a lentivirus having an integrase that lacks the capacity to integrate the viral genome into the genome of the host cells. Integration-incompetent viral vectors have been described in patent application WO 2006/010834, which is herein incorporated by reference in its entirety.

Illustrative mutations in the HIV-1 pol gene suitable to reduce integrase activity include, but are not limited to: H12N, H12C, H16C, H16V, S81 R, D41A, K42A, H51A, Q53C, D55V, D64E, D64V, E69A, K71A, E85A, E87A, D116N, D1161, D116A, N120G, N1201, N120E, E152G, E152A, D35E, K156E, K156A, E157A, K159E, K159A, K160A, R166A, D167A, E170A, H171A, K173A, K186Q, K186T, K188T, E198A, R199c, R199T, R199A, D202A, K211A, Q214L, Q216L. Q221 L, W235F, W235E, K236S, K236A, K246A, G247W, D253A, R262A, R263A and K264H.

In one embodiment, the HIV-1 integrase deficient pol gene comprises a D64V, D116I, D116A, E152G, or E152A mutation; D64V, D116I, and E152G mutations; or D64V, D116A, and E152A mutations.

In one embodiment, the HIV-1 integrase deficient pol gene comprises a D64V mutation.

The term “long terminal repeat (LTR)” refers to domains of base pairs located at the ends of retroviral DNAs which, in their natural sequence context, are direct repeats and contain U3, R and U5 regions.

As used herein, the term “FLAP element” or “cPPT/FLAP” refers to a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 101:173. In another embodiment, a lentiviral vector contains a FLAP element with one or more mutations in the cPPT and/or CTS elements. In yet another embodiment, a lentiviral vector comprises either a cPPT or CTS element. In yet another embodiment, a lentiviral vector does not comprise a cPPT or CTS element.

As used herein, the term “packaging signal” or “packaging sequence” refers to psi [Ψ] sequences located within the retroviral genome which are required for insertion of the viral RNA into the viral capsid or particle, see e.g., Clever et al., 1995. J. of Virology, Vol. 69, No. 4; pp. 2101-2109.

The term “export element” refers to a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus post-transcriptional regulatory element (HPRE).

In particular embodiments, expression of heterologous sequences in viral vectors is increased by incorporating posttranscriptional regulatory elements, efficient polyadenylation sites, and optionally, transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766).

Lentiviral vectors preferably contain several safety enhancements as a result of modifying the LTRs. “Self-inactivating” (SIN) vectors refers to replication-defective vectors, e.g., in which the right (3′) LTR enhancer-promoter region, known as the U3 region, has been modified (e.g., by deletion or substitution) to prevent viral transcription beyond the first round of viral replication. An additional safety enhancement is provided by replacing the U3 region of the 5′ LTR with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters.

The terms “pseudotype” or “pseudotyping” as used herein, refer to a virus that has viral envelope proteins that have been substituted with those of another virus possessing preferable characteristics. For example, HIV can be pseudotyped with vesicular stomatitis virus G-protein (VSV-G) envelope proteins, which allows HIV to infect a wider range of cells because HIV envelope proteins (encoded by the env gene) normally target the virus to CD4⁺ presenting cells.

In certain embodiments, lentiviral vectors are produced according to known methods. See e.g., Kutner et al., BMC Biotechnol. 2009;9:10. doi: 10.1186/1472-6750-9-10; Kutner et al. Nat. Protoc. 2009;4(4):495-505. doi: 10.1038/nprot.2009.22.

According to certain specific embodiments contemplated herein, most or all of the viral vector backbone sequences are derived from a lentivirus, e.g., HIV-1. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. Moreover, a variety of lentiviral vectors are known in the art, see Naldini et al., (1996a, 1996b, and 1998); Zufferey et al., (1997); Dull et al., 1998, U.S. Pat. Nos. 6,013,516; and 5,994,136, many of which may be adapted to produce a viral vector or transfer plasmid contemplated herein.

In various embodiments the lentiviral vector is an AnkT9W vector, a T9Ank2W vector, a TNS9 vector, a TNS9.3 vector, a TNS9.3.55 vector, a lentiglobin HPV569 vector, a lentiglobin BB305 vector, a BG-1 vector, a BGM-1 vector, a mLARβΔγV5 vector, a GLOBE vector, a G-GLOBE vector, a βAS3-FB vector, a V5 vector, a V5m3 vector, a V5m3-400 vector, a G9 vector, or a derivative thereof. In some embodiments, the lentiviral vector is an AnkT9W vector or a derivative thereof. In some embodiments, the lentiviral vector is a T9Ank2W vector or a derivative thereof. In some embodiments, the lentiviral vector is a TNS9 vector or a derivative thereof. In some embodiments, the lentiviral vector is a TNS9.3 vector or a derivative thereof. In some embodiments, the lentiviral vector is a TNS9.3.55 vector or a derivative thereof. In some embodiments, the lentiviral vector is a lentiglobin HPV569 vector or a derivative thereof. In some embodiments, the lentiviral vector is a lentiglobin BB305 vector or a derivative thereof. In some embodiments, the lentiviral vector is a BG-1 vector or a derivative thereof. In some embodiments, the lentiviral vector is a BGM-1 vector or a derivative thereof. In some embodiments, the lentiviral vector is a mLARβΔγV5 vector, or a derivative thereof. In some embodiments, the lentiviral vector is a GLOBE vector or a derivative thereof. In some embodiments, the lentiviral vector is a G-GLOBE vector or a derivative thereof. In some embodiments, the lentiviral vector is a βAS3-FB vector or a derivative thereof. In some embodiments, the lentiviral vector is a V5 vector. In some embodiments, the lentiviral vector is a V5m3 vector, or a derivative thereof. In some embodiments, the lentiviral vector is a V5m3-400 vector, or a derivative thereof. In some embodiments, the lentiviral vector is a G9 vector, or a derivative thereof.

In various embodiments, one or more polynucleotides or vectors encoding a β-globin are introduced into a cell (e.g., K562 cells) by transducing the cell with an adenovirus comprising the one or more polynucleotides.

Adenoviral based vectors are capable of very high transduction efficiency in many cell types and do not require cell division. With such vectors, high titer and high levels of expression have been obtained. This vector can be produced in large quantities in a relatively simple system. Most adenovirus vectors are engineered such that a transgene replaces the Ad Ela, Elb, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney and muscle. Conventional Ad vectors have a large carrying capacity.

Generation and propagation of the current adenovirus vectors, which are replication deficient, may utilize a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones & Shenk, 1978), the current adenovirus vectors, with the help of 293 cells, carry foreign DNA in either the E1, the D3 or both regions (Graham & Prevec, 1991). Adenovirus vectors have been used in eukaryotic gene expression (Levrero et al., 1991; Gomez-Foix et al., 1992) and vaccine development (Grunhaus & Horwitz, 1992; Graham & Prevec, 1992). Studies in administering recombinant adenovirus to different tissues include trachea instillation (Rosenfeld et al., 1991; Rosenfeld et al., 1992), muscle injection (Ragot et al., 1993), peripheral intravenous injections (Herz & Gerard, 1993) and stereotactic inoculation into the brain (Le Gal La Salle et al., 1993). An example of the use of an Ad vector in a clinical trial involved polynucleotide therapy for antitumor immunization with intramuscular injection (Sterman et al., Hum. Gene Ther. 7:1083-9 (1998)).

In various embodiments, one or more polynucleotides or vectors encoding β-globin are introduced into a cell by transducing the cell (e.g., K562 cell) with a herpes simplex virus, e.g., HSV-1, HSV-2, comprising the one or more polynucleotides.

The mature HSV virion consists of an enveloped icosahedral capsid with a viral genome consisting of a linear double-stranded DNA molecule that is 152 kb. In one embodiment, the HSV based viral vector is deficient in one or more essential or non-essential HSV genes. In one embodiment, the HSV based viral vector is replication deficient. Most replication deficient HSV vectors contain a deletion to remove one or more intermediate-early, early, or late HSV genes to prevent replication. For example, the HSV vector may be deficient in an immediate early gene selected from the group consisting of: ICP4, ICP22, ICP27, ICP47, and a combination thereof. Advantages of the HSV vector are its ability to enter a latent stage that can result in long-term DNA expression and its large viral DNA genome that can accommodate exogenous DNA inserts of up to 25 kb. HSV-based vectors are described in, for example, U.S. Pat. Nos. 5,837,532, 5,846,782, and 5,804,413, and International Patent Applications WO 91/02788, WO 96/04394, WO 98/15637, and WO 99/06583, each of which are incorporated by reference herein in its entirety.

In certain embodiments, the cells are transduced with a vector as described herein in the presence of a polycationic polymer. In some embodiments, the polycationic polymer is polybrene, protamine sulfate, polyethylenimine, or a polyethylene glycol/poly-L-lysine block copolymer. In some embodiments, the cells are transduced in the presence of polybrene. In some embodiments, the cells are transduced in the presence of about 2 μg/ml polybrene. In some embodiments, the cells are transduced in the presence of about 3 μg/ml polybrene. In some embodiments, the cells are transduced in the presence of about 4 μg/ml polybrene. In some embodiments, the cells are transduced in the presence of about 5 μg/ml polybrene. In some embodiments, the cells are transduced in the presence of about 6 μg/ml polybrene. In some embodiments, the cells are transduced in the presence of about 7 μg/ml polybrene. In some embodiments, the cells are transduced in the presence of about 8 μg/ml polybrene. In some embodiments, the cells are transduced in the presence of about 2 μg/ml to about 8 μg/ml polybrene. In some embodiments, the cells are transduced in the presence of about 3 μg/ml to about 8 μg/ml polybrene. In some embodiments, the cells are transduced in the presence of about 4 μg/ml to about 8 μg/ml polybrene. In some embodiments, the cells are transduced in the presence of about 5 μg/ml to about 8 μg/ml polybrene. In some embodiments, the cells are transduced in the presence of about 6 μg/ml to about 8 μg/ml polybrene. In some embodiments, the cells are transduced in the presence of about 7 μg/ml to about 8 μg/ml polybrene.

Endonucleases may be used to introduce a DSB in a target sequence; the DSB may be repaired through homology directed repair (HDR) mechanisms in the presence of one or more donor repair templates. In some embodiments, the donor repair template is used to insert a sequence into the genome. In particular preferred embodiments, the donor repair template is used to insert a polynucleotide sequence encoding a globin gene/protein (e.g., a therapeutic β-globin).

As contemplated elsewhere herein, the endonuclease may be introduced by viral or non-viral methods. In some embodiments, an endonuclease polypeptide is introduced into a cell. In some embodiments, a polynucleotide encoding an endonuclease is introduced into a cell. In some embodiments, the endonuclease or polynucleotide encoding the endonuclease is introduced in the cells by viral or non-viral methods as contemplated herein, e.g., transfection, transduction, or electroporation.

In various embodiments, the cells are modified to express a globin gene, wherein the modifying comprises introducing into the cells (a) an endonuclease or polynucleotide encoding an endonuclease, and (b) a donor repair template encoding a β-globin.

In particular embodiments, the endonuclease is selected from the group consisting of: a homing endonuclease, or functional variant thereof; a megaTAL, or functional variant thereof; a CRISPR-associated nuclease, or functional variant thereof; a zinc-finger nuclease, or functional variant thereof; and a transcription activator-like effector nuclease (TALEN), or functional variant thereof.

As contemplated elsewhere herein, a donor repair template may be is introduced into a cell by viral or non-viral methods. In some embodiments, the donor repair template is introduced by transducing the cell with an adeno-associated virus (AAV), retrovirus, e.g., lentivirus, IDLV, etc., herpes simplex virus, adenovirus, or vaccinia virus vector comprising the donor repair template.

In particular embodiments, the donor repair template comprises one or more homology arms that flank a double strand break site of the endonuclease. As used herein, the term “homology arms” refers to a nucleic acid sequence in a donor repair template that is identical, or nearly identical, to DNA sequence flanking the DNA break introduced by the nuclease at a target site. In one embodiment, the donor repair template comprises a 5′ homology arm that comprises a nucleic acid sequence that is identical or nearly identical to the DNA sequence 5′ of the DNA break site. In one embodiment, the donor repair template comprises a 3′ homology arm that comprises a nucleic acid sequence that is identical or nearly identical to the DNA sequence 3′ of the DNA break site. In a preferred embodiment, the donor repair template comprises a 5′ homology arm and a 3′ homology arm. The donor repair template may comprise homology to the genome sequence immediately adjacent to the DSB site, or homology to the genomic sequence within any number of base pairs from the DSB site. In one embodiment, the donor repair template comprises a nucleic acid sequence that is homologous to a genomic sequence about 5 bp, about 10 bp, about 25 bp, about 50 bp, about 100 bp, about 250 bp, about 500 bp, about 1000 bp, about 2500 bp, about 5000 bp, about 10000 bp or more, including any intervening length of homologous sequence.

Illustrative examples of suitable lengths of homology arms contemplated in particular embodiments, may be independently selected, and include but are not limited to: about 100 bp, about 200 bp, about 300 bp, about 400 bp, about 500 bp, about 600 bp, about 700 bp, about 800 bp, about 900 bp, about 1000 bp, about 1100 bp, about 1200 bp, about 1300 bp, about 1400 bp, about 1500 bp, about 1600 bp, about 1700 bp, about 1800 bp, about 1900 bp, about 2000 bp, about 2100 bp, about 2200 bp, about 2300 bp, about 2400 bp, about 2500 bp, about 2600 bp, about 2700 bp, about 2800 bp, about 2900 bp, or about 3000 bp, or longer homology arms, including all intervening lengths of homology arms.

Additional illustrative examples of suitable homology arm lengths include, but are not limited to: about 100 bp to about 3000 bp, about 200 bp to about 3000 bp, about 300 bp to about 3000 bp, about 400 bp to about 3000 bp, about 500 bp to about 3000 bp, about 500 bp to about 2500 bp, about 500 bp to about 2000 bp, about 750 bp to about 2000 bp, about 750 bp to about 1500 bp, or about 1000 bp to about 1500 bp, including all intervening lengths of homology arms.

In a particular embodiment, the lengths of the 5′ and 3′ homology arms are independently selected from about 500 bp to about 1500 bp. In one embodiment, the 5 ‘homology arm is about 1500 bp and the 3’ homology arm is about 1000 bp. In one embodiment, the 5 homology arm is between about 200 bp to about 600 bp and the 3′ homology arm is between about 200 bp to about 600 bp. In one embodiment, the 5 ‘homology arm is about 200 bp and the 3’ homology arm is about 200 bp. In one embodiment, the 5 ‘homology arm is about 300 bp and the 3’ homology arm is about 300 bp. In one embodiment, the 5 ‘homology arm is about 400 bp and the 3’ homology arm is about 400 bp. In one embodiment, the 5 ‘homology arm is about 500 bp and the 3’ homology arm is about 500 bp. In one embodiment, the 5 ‘homology arm is about 600 bp and the 3’ homology arm is about 600 bp.

In various embodiments, the donor repair template is introduced by transfection, transduction, or electroporation.

### E. Chromatography

In various embodiments, the methods/process described herein use liquid chromatography to separate the different forms hemoglobin, e.g., HbF and HbA. As used herein, the terms “liquid chromatography”, “LC” refers to a process wherein components of a sample are separated based on interactions of the sample with a mobile phase (i.e., a liquid mobile phase) and a stationary phase. The liquid mobile phase passed down through the solid stationary phase (along with the separated components), into a detection unit for proper detection and/or quantitation. Non-limiting examples of liquid chromatography processes include HPLC and UPLC.

The terms “high-performance liquid chromatography,” “high-pressure liquid chromatography,” and “HPLC”, refer to a liquid chromatography technique that uses pumps to pass a pressurized liquid solvent containing the sample mixture through a column filled with a solid/stationary adsorbent material or substrate. Typically, HPLC systems operate at a pressure of about 500-6000 psi.

The terms “ultra-high performance liquid chromatography,” “UHPLC,” “ultra-performance liquid chromatography,” and “UPLC” refer to a liquid chromatography technique that uses pumps to pass a highly pressurized liquid solvent containing the sample mixture through a column filled with a solid/stationary adsorbent material or substrate. However, in contrast to HPLC systems, UPLC systems operate at a pressure of greater than 6000 psi (e.g., about 15000 psi). UHPLC and UPLC systems operate at higher pressures because, generally, these systems use smaller particles as the solid phase substrate/material (e.g., particle sizes of less than 2 micron), as compared to HPLC systems (e.g., particle sizes of about 5 micron). Additionally, the inner diameter of UHPLC columns is generally smaller than HPLC systems. For example, about 2.5 mm to about 5 mm for HPLC columns, and about 2.1 mm or less for UPLC columns (e.g., 1 mm).

In various embodiments, the methods describe herein comprise a step of analyzing a cell lysate with a liquid chromatography system comprising passing the cell lysate through chromatographic column. In some embodiments, the method comprises HPLC. In some embodiments, the method comprises UHPLC or UPLC. In some embodiments the liquid chromatography is an ion-exchange liquid chromatography. In particular embodiments, the HPLC, UHPLC, or UPLC, is an ion-exchange HPLC, UHPLC, or UPLC.

Whether HPLC, UPLC, or UHPLC, the sample containing molecules of interest is analyzed with a detector that detects the abundance of the molecules and shows their retention on the chromatographic column in relation to the elapsed time (retention time). Retention times vary depending on the interactions between the stationary phase, the molecules being analyzed, diluent, and the mobile phase solvent(s) used. A sample containing the metabolites is injected into the mobile phase manually or by an automated autosampler. The polarity of the metabolites, the stationary phase of the column(s) used and the mobile phase(s) determine the retention time of the metabolite as well as its separation from interferences and extent of quantifiability.

Accordingly, in various embodiments disclosed herein, after chromatographic separation, the hemoglobin proteins/complexes can be detected by detecting the associated heme molecules/groups by measuring ultraviolet (UV) light absorbance at 418 nm. In various embodiments, the chromatography comprises a UV detector. In some embodiments, the UV detector is a tunable ultraviolet (TUV) detector. In some embodiments, the UV detector is a photodiode array ultraviolet (PDA UV) detector.

The terms “ion chromatography.” “ion-exchange chromatography”, “ion-exchange liquid chromatography.” and “IEX” refer to chromatography methods that separate ions and polar molecules based on their affinity to charged sites bound to the solid/stationary phase (e.g., ion-exchangers). Illustrative ion-exchangers include, but are not limited to, polystyrene resins, cellulose and dextran ion exchangers (gels), and controlled-pore glass or porous silica.

Generally, there are two types of IEX, anion-exchange and cation-exchange. In cation-exchange chromatography the stationary phase is negatively charged and the molecules to be separated are positively charged (i.e., the pH for chromatography is less than the molecule pI). In anion-exchange chromatography the stationary phase is positively charged and the molecules to be separated are negatively charged (i.e., the pH for chromatography is greater than the molecule pI). In either case, the charge molecules to be separated (e.g., proteins, amino acids, and peptides) bind to sites which are oppositely charged by forming ionic bonds to the insoluble solid/stationary phase. The bound molecules are then eluted and collected using an eluant having a higher concentration of ions (anions or cations) through the column or by changing pH of the column. For example, in cation exchange chromatography, a positively charged molecule could be displaced from the solid/stationary phase by the addition of positively charged sodium ions.

In various embodiments, the methods describe herein comprise, IEX chromatography to separate hemoglobin proteins (e.g., HbF and HbA). In some embodiments, the IEX chromatography is IEX HPLC. In some embodiments, the IEX chromatography is IEX UPLC. In some embodiments, the IEX chromatography is IEX UHLPC.

A chromatographic column (e.g., an IEX liquid chromatography column) typically includes two ports, one inlet port for receiving a sample and one outlet port for discharging an eluent that may or may not include the sample. Columns suitable for liquid chromatography comprise a solid phase comprising packing materials/substrates comprising very small and usually spherical particles, e.g., silica particles, having a diameter of 3-50 microns and a pore size of about 60-1500 angstroms. Other suitable packing materials/substrates include, but are not limited to, polystyrene resins, cellulose and dextran ion exchangers (gels), and controlled-pore glass or porous silica. In various embodiments, the solid phase may also comprise a molecule (e.g., amino acid) that enables ion change or ion pairing (e.g., aspartic acid chains or sulfonic acid ligands).

Accordingly, in various embodiments, the column comprises a solid phase comprising aspartic acid chains covalently linked to a substrate. In certain embodiments, the column comprises a solid phase comprising sulfonic acid ligands covalently lined to a substrate. In some embodiments, the substrate is a polymer substrate. In particular embodiments, the substrate is a silica substrate. In some embodiments, the substrate has a particle size of about 5 μm. In some embodiments, the substrate has a particle size of about 4 μm. In some embodiments, the substrate has a particle size of about 3 μm. In some embodiments, the substrate has a particle size of about 2 μm. In some embodiments, the substrate has a particle size of about 1 μm.

In some embodiments, the substrate has a pore size of about 60 angstroms. In some embodiments, the substrate has a pore size of about 100 angstroms. In some embodiments, the substrate has a pore size of about 200 angstroms. In some embodiments, the substrate has a pore size of about 300 angstroms. In some embodiments, the substrate has a pore size of about 400 angstroms. In some embodiments, the substrate has a pore size of about 500 angstroms. In some embodiments, the substrate has a pore size of about 600 angstroms. In some embodiments, the substrate has a pore size of about 700 angstroms. In some embodiments, the substrate has a pore size of about 800 angstroms. In some embodiments, the substrate has a pore size of about 900 angstroms. In some embodiments, the substrate has a pore size of about 1000 angstroms. In some embodiments, the substrate has a pore size of about 1500 angstroms.

The internal diameter of a liquid chromatography column may vary depending on the application or method used (e.g., HPLC or UPLC). The internal diameter for HPLC columns are typically larger than for UHPLC/UPLC columns. For example, the internal diameter for HPLC columns may vary between about 2.5 mm to about 5 mm, while the internal diameter for UHPLC columns are typically less than 2.5 mm (e.g., about 2.1 mm or less). Accordingly, in some embodiments described herein, the column comprises an internal diameter of about 5 mm or less. In some embodiments, the column comprises an internal diameter of about 4 mm or less. In some embodiments, the column comprises an internal diameter of about 3 mm or less. In some embodiments, the column comprises an internal diameter of about 2.5 mm or less. In some embodiments, the column comprises an internal diameter of about 2.1 mm or less. In some embodiments, the column comprises an internal diameter of about 2 mm or less. In some embodiments, the column comprises an internal diameter of about 1 mm or less.

In some embodiments, the column comprises an internal diameter of about 1 mm to about 5 mm. In some embodiments, the column comprises an internal diameter of about 1 mm to about 4 mm. In some embodiments, the column comprises an internal diameter of about 1 mm to about 3 mm. In some embodiments, the column comprises an internal diameter of about 1 mm to about 2.5 mm. In some embodiments, the column comprises an internal diameter of about 1 mm to about 2.1 mm.

In particular embodiments, the column comprises sulfonic acid ligands linked to a polymer substrate having a particle size of about 3 μm, and an internal diameter of about 2.1 mm. In particular embodiments, the column comprises aspartic acid chains covalently linked to a silica substrate having a particle size of about 5 μm and a pore diameter of about 1000 angstroms, wherein the column has an internal diameter of about 2.1 mm. In particular embodiments, the column comprises aspartic acid chains covalently linked to a silica substrate having a particle size of about 5 μm and a pore diameter of about 1000 angstroms, wherein the column has an internal diameter of about 1 mm. In some embodiments the column length is 150 mm. In some embodiments, the column length is 100 mm.

In some embodiments, the liquid chromatography comprises a liquid-based mobile phase. As described herein, the mobile phase may comprise different solvents or solvent mixtures for eluting the hemoglobin proteins/complexes. For example, liquid chromatography may be performed using a gradient mode with differing amounts of solvents in the mixture, an isocratic mode with continuously fixed amounts of solvents in the mixture or a partially isocratic, partially gradient mixed mode. Suitable solvents and solvent mixtures include sodium or lithium buffers (for cation exchange HPLC) or acetonitrile (for reverse phase HPLC). Other illustrative mobile phases comprise a Tris buffer, KCN, and/or NaCl. In some embodiments, the mobile phase comprises triethylamine (TEA).

In some embodiments, the liquid chromatography comprises a liquid-based first and second mobile phases. In some embodiments, the first mobile phase comprises the sample to be separated and is formulated to promote binding to the solid phase. In some embodiments, the second mobile phase is formulated to drive sample separation when the components (hemoglobin proteins/complexes) are eluted.

In some embodiments, the first mobile phase comprises a Tris buffer and KCN. In some embodiments, the second mobile phase comprises Tris buffer, KCN, and NaCl. In some embodiments, the first and/or second mobile phase comprises about 38 mM to about 42 mM Tris buffer. In some embodiments, the first and/or second mobile phase comprises about 38 mM Tris buffer. In some embodiments, the first and/or second mobile phase comprises about 40 mM Tris buffer. In some embodiments, the first and/or second mobile phase comprises about 42 mM Tris buffer. In some embodiments, the first and/or second mobile phase comprises about 2.9 mM to about 3.1 KCN. In some embodiments, the first and/or second mobile phase comprises about 2.9 mM KCN. In some embodiments, the first and/or second mobile phase comprises about 3.0 mM KCN. In some embodiments, the first and/or second mobile phase comprises about 3.1 mM KCN. In some embodiments, the second mobile phase comprises about 0.1 M to about 0.3 M NaCl. In some embodiments, the second mobile phase comprises about 0.1 M NaCl. In some embodiments, the second mobile phase comprises about 0.19 M NaCl. In some embodiments, the second mobile phase comprises about 0.20 M NaCl. In some embodiments, the second mobile phase comprises about 0.21 M NaCl. In some embodiments, the second mobile phase comprises about 0.3 M NaCl.

In some embodiments, the liquid chromatography comprises a mobile phase gradient as shown in Tables 1 and 2.

TABLE 1
Illustrative mobile phase gradient 1

| Time (min) | First Mobile Phase (%) | Second Mobile Phase (%) |
|---|---|---|
| 0 | 100 | 0 |
| 6 | 0 | 100 |
| 7 | 0 | 100 |
| 11 | 100 | 0 |
| 13 | 100 | 0 |

TABLE 2
Illustrative mobile phase gradient 2

| Time (min) | First Mobile Phase (%) | Second Mobile Phase (%) |
|---|---|---|
| 0 | 100 | 0 |
| 2.5 | 50 | 50 |
| 6 | 0 | 100 |
| 7 | 0 | 100 |
| 11 | 100 | 0 |
| 13 | 100 | 0 |

In various embodiments, the first and/or second mobile phase has a pH of about 6.0 to about 7.0. In various embodiments, the first and/or second mobile phase has a pH of about 6.4 to about 6.6. In some embodiments, the first and/or second mobile phase has a pH of about 6.0. In some embodiments, the first and/or second mobile phase has a pH of about 6.1. In some embodiments, the first and/or second mobile phase has a pH of about 6.2. In some embodiments, the first and/or second mobile phase has a pH of about 6.3. In some embodiments, the first and/or second mobile phase has a pH of about 6.4. In some embodiments, the first and/or second mobile phase has a pH of about 6.5. In some embodiments, the first and/or second mobile phase has a pH of about 6.6. In some embodiments, the first and/or second mobile phase has a pH of about 6.7. In some embodiments, the first and/or second mobile phase has a pH of about 6.8. In some embodiments, the first and/or second mobile phase has a pH of about 6.9. In some embodiments, the first and/or second mobile phase has a pH of about 7.0.

In some embodiments, the first and/or second mobile phase has a pH of 6.0±0.1. In some embodiments, the first and/or second mobile phase has a pH of about 6.1±0.1. In some embodiments, the first and/or second mobile phase has a pH of about 6.2±0.1. In some embodiments, the first and/or second mobile phase has a pH of about 6.3±0.1. In some embodiments, the first and/or second mobile phase has a pH of about 6.4±0.1. In some embodiments, the first and/or second mobile phase has a pH of about 6.5±0.1. In some embodiments, the first and/or second mobile phase has a pH of about 6.6±0.1. In some embodiments, the first and/or second mobile phase has a pH of about 6.7±0.1. In some embodiments, the first and/or second mobile phase has a pH of about 6.8±0.1. In some embodiments, the first and/or second mobile phase has a pH of about 6.9±0.1. In some embodiments, the first and/or second mobile phase has a pH of about 7.0±0.1.

In some embodiments, the column temperature is about 25° C. to about 30° C. In some embodiments, the column temperature is about 25° C. In some embodiments, the column temperature is about 26° C. In some embodiments, the column temperature is about 27° C. In some embodiments, the column temperature is about 28° C. In some embodiments, the column temperature is about 29° C. In some embodiments, the column temperature is about 30° C.

Hemoglobin separation using liquid chromatography (LC) as described herein may be performed with any commercially available LC apparatus/system using automated or manual sample injection and adjustable, consistent and reproducible solvent flow rates. Illustrative systems known in the art, include by are not limited to Shimadzu LC (HPLC or UHPLC), Waters Acquity™, Agilent (e.g., Infinity II system), and AKTA™ (e.g., AKTA Pure system).

All publications, patent applications, and issued patents cited in this specification are herein incorporated by reference as if each individual publication, patent application, or issued patent were specifically and individually indicated to be incorporated by reference.

Although the foregoing embodiments have been described in some detail by way of illustration and example for purposes of clarity of understanding, it will be readily apparent to one of ordinary skill in the art in light of the teachings contemplated herein that certain changes and modifications may be made thereto without departing from the spirit or scope of the appended claims. The following examples are provided by way of illustration only and not by way of limitation. Those of skill in the art will readily recognize a variety of noncritical parameters that could be changed or modified to yield essentially similar results.

### EXAMPLES

### Example 1

### Culture Density and Transduction Conditions

In order to develop a method for the measurement of HbA in lentiviral transduced cells and the functional potency of lentiviral particles encoding a β-globin (e.g., LentiGlobin BB305 which encodes β^{A-T87Q} globin) using K562 cells, an experiment to define the critical culture and transduction parameters was conducted. Cells from a K562 master cell bank were seeded in a 12-well plate at concentrations of 0.5×10⁶, 1.0×10⁶, and 3.0×10⁶ cells/mL (1 mL total volume). Following plating, cells were transduced with adherent lentiviral vector (LVV) encoding Hemoglobin A^{T87Q} at MOIs of 5, 10, and 20, with and without 8 μg/mL polybrene. Samples were taken at 48 h and 72 h post-transduction and analyzed by Shimadzu HPLC using a PolyLC PolyCAT A 200×2.1 mm, 5 μm, 1000 A column.

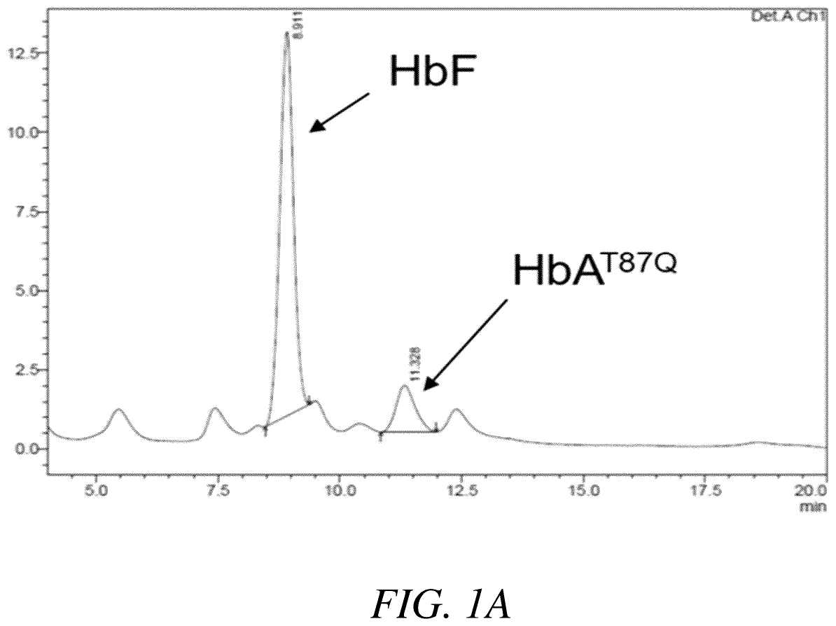

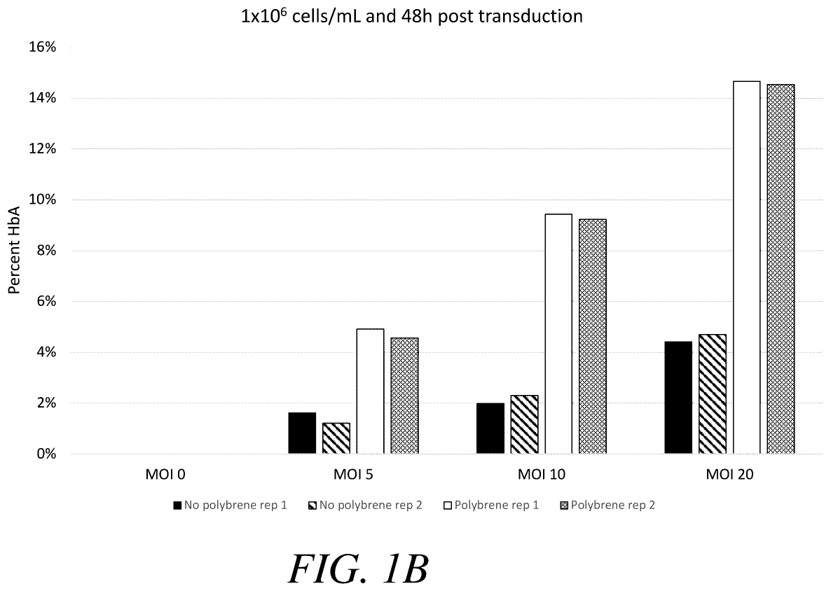

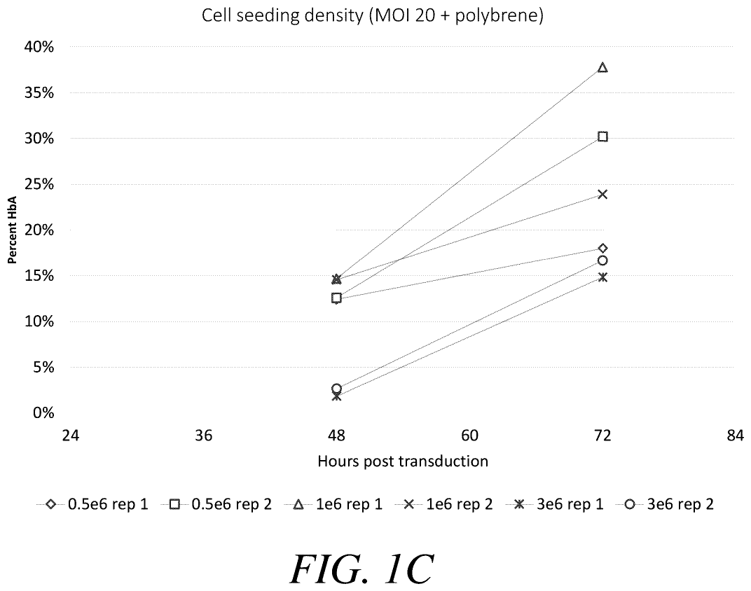

The results of the experiment are presented in FIGS. 1A and 1B. A representative chromatogram of samples is shown FIG. 1A and demonstrates an LVV-derived Hemoglobin A (HbA) peak and the endogenously expressed Hemoglobin F (HbF) peak. Results are calculated as percent HbA (% HbA) based on the HbA peak area relative to the sum of HbA and HbF peak areas. An MOI-dependent increase in % HbA is observed in transduced cells, with the use of polybrene improving the transduction efficiency and resulting % HbA (FIG. 1B). FIG. 1C shows variable % HbA as a function of starting cell density, with about 1×10⁶ cells/mL demonstrating higher and more consistent time dependent increases in % HbA.

### Example 2

### MOI Analysis

To assess harvest time and MOI, 1×10⁶ K562 cells were transduced at MOIs 5, 10, 20, 40 and 100 in triplicate using two different LVV vector lots and analyzed out to nine days post-transduction. Transduction was performed in T25 flasks to facilitate the repeated sampling required for the time course analysis, and samples were analyzed by HPLC using a PolyLC PolyCAT A 200×2.1 mm, 5 μm, 1000 A column.

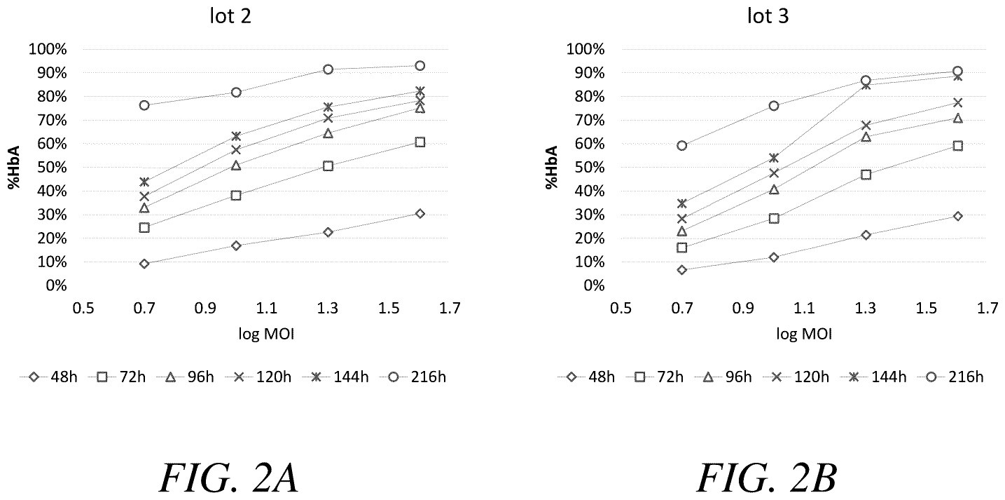

The data was evaluated by comparing R² values to identify a linear fit model that best predicted the dose-response data. Linear fits were evaluated using both HbA area under the curve and % HbA readouts, over the MOI ranges 5-100 and 5-40, and using MOI and the log₁₀ transformed MOI. The results are presented in Table 3 and show that a log₁₀ transformation of the MOI resulted in improved fit of the dose-response, while restricting the dose to a maximum of the log₁₀ of MOI 40 further improved the fit. In yet other experiments, a range of 10-30 MOI was also sufficient to improve the fit and, in some instances, is preferred. Use of % HbA did not significantly improve the average of the fits relative to the raw HbA area under the curve values, however % HbA markedly improved the minimum R² value observed and the precision of the readout across triplicate transductions (Table 3 and Table 4). The percent HbA was plotted against the log₁₀ MOI for each harvest time and the curves are presented in FIGS. 2A and 2B.

TABLE 3
Comparison of R² values of linear fit models

| | | HbA AUC | | | | % HbA | | | |
|---|---|---|---|---|---|---|---|---|---|
| | Time | MOI range | | log MOI range | | MOI range | | log MOI range | |
| | (h) | 5-100 | 5-40 | 5-100 | 5-40 | 5-100 | 5-40 | 5-100 | 5-40 |
| Lot 2 | 48 | 0.79 | 0.93 | 0.99 | 1.00 | 0.77 | 0.93 | 0.99 | 1.00 |
| | 72 | 0.50 | 0.73 | 0.85 | 0.93 | 0.68 | 0.88 | 0.96 | 1.00 |
| | 96 | 0.66 | 0.97 | 0.93 | 0.97 | 0.64 | 0.85 | 0.94 | 0.99 |
| | 120 | 0.85 | 0.68 | 0.95 | 0.90 | 0.57 | 0.78 | 0.90 | 0.96 |
| | 144 | 0.68 | 0.91 | 0.95 | 0.98 | 0.57 | 0.77 | 0.90 | 0.95 |
| | 216 | 0.78 | 0.90 | 0.99 | 1.00 | 0.40 | 0.78 | 0.77 | 0.94 |
| Lot 3 | 48 | 0.85 | 0.62 | 0.91 | 0.79 | 0.70 | 0.94 | 0.96 | 0.99 |
| | 72 | 0.84 | 0.99 | 0.98 | 0.95 | 0.64 | 0.91 | 0.94 | 0.99 |
| | 96 | 0.69 | 0.71 | 0.94 | 0.92 | 0.59 | 0.82 | 0.91 | 0.97 |
| | 120 | 0.29 | 0.45 | 0.61 | 0.71 | 0.58 | 0.83 | 0.91 | 0.98 |
| | 144 | 0.61 | 0.99 | 0.89 | 0.96 | 0.50 | 0.76 | 0.84 | 0.93 |
| | 216 | 0.55 | 0.87 | 0.89 | 0.99 | 0.56 | 0.72 | 0.88 | 0.93 |

| | | | | | | | | |
|---|---|---|---|---|---|---|---|---|
| Average | 0.67 | 0.81 | 0.91 | 0.93 | 0.60 | 0.83 | 0.91 | 0.97 |
| Std. Dev. | 0.17 | 0.17 | 0.10 | 0.09 | 0.10 | 0.07 | 0.06 | 0.03 |
| Minimum | 0.29 | 0.45 | 0.61 | 0.71 | 0.40 | 0.72 | 0.77 | 0.93 |

TABLE 4
Precision of Triplicate Transductions (% CV)

| | | HbA AUC | | | | | % HbA | | | | |
|---|---|---|---|---|---|---|---|---|---|---|---|
| | Time | MOI | MOI | MOI | MOI | MOI | MOI | MOI | MOI | MOI | MOI |
| | (h) | 5 | 10 | 20 | 40 | 100 | 5 | 10 | 20 | 40 | 100 |
| Lot 2 | 48 | 35% | 12% | 7% | 10% | 7% | 10% | 2% | 6% | 2% | 1% |
| | 72 | 4% | 5% | 1% | 14% | 13% | 2% | 1% | 4% | 1% | 1% |
| | 96 | 10% | 9% | 5% | 13% | 41% | 2% | 3% | 4% | 1% | 1% |
| | 120 | 5% | 14% | 15% | 39% | 18% | 0% | 0% | 2% | 0% | 1% |
| | 144 | 13% | 21% | 9% | 10% | 10% | 2% | 2% | 2% | 1% | 0% |
| | 216 | 23% | 4% | 27% | 3% | 2% | 12% | 1% | 0% | 0% | 0% |
| Lot 3 | 48 | 11% | 27% | 43% | 34% | 49% | 5% | 3% | 8% | 3% | 3% |
| | 72 | 7% | 21% | 20% | 8% | 17% | 4% | 3% | 2% | 1% | 2% |
| | 96 | 0% | 5% | 8% | 25% | 42% | 10% | 5% | 2% | 2% | 2% |
| | 120 | 12% | 12% | 9% | 13% | 33% | 4% | 2% | 0% | 1% | 1% |
| | 144 | 7% | 21% | 13% | 9% | 40% | 2% | 4% | 1% | 0% | 0% |
| | 216 | 7% | 5% | 14% | 12% | 22% | 4% | 2% | 4% | 0% | 0% |

| | | | | | | | | | | |
|---|---|---|---|---|---|---|---|---|---|---|
| Maximum | 35% | 27% | 43% | 39% | 49% | 12% | 5% | 8% | 3% | 3% |

### Example 3

### Harvest Time Analysis

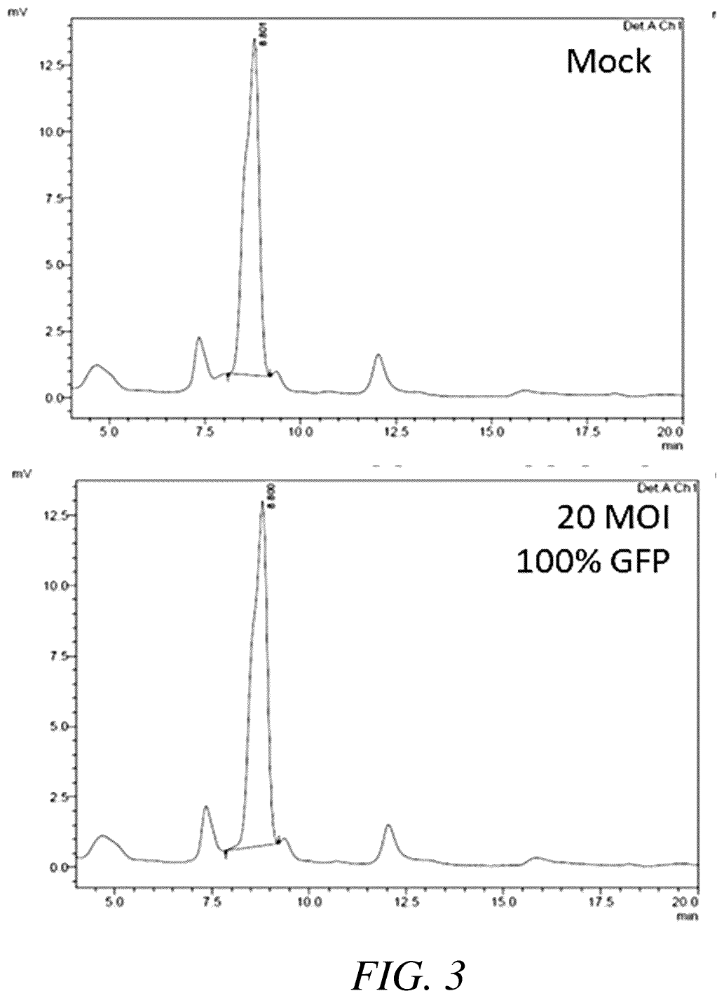

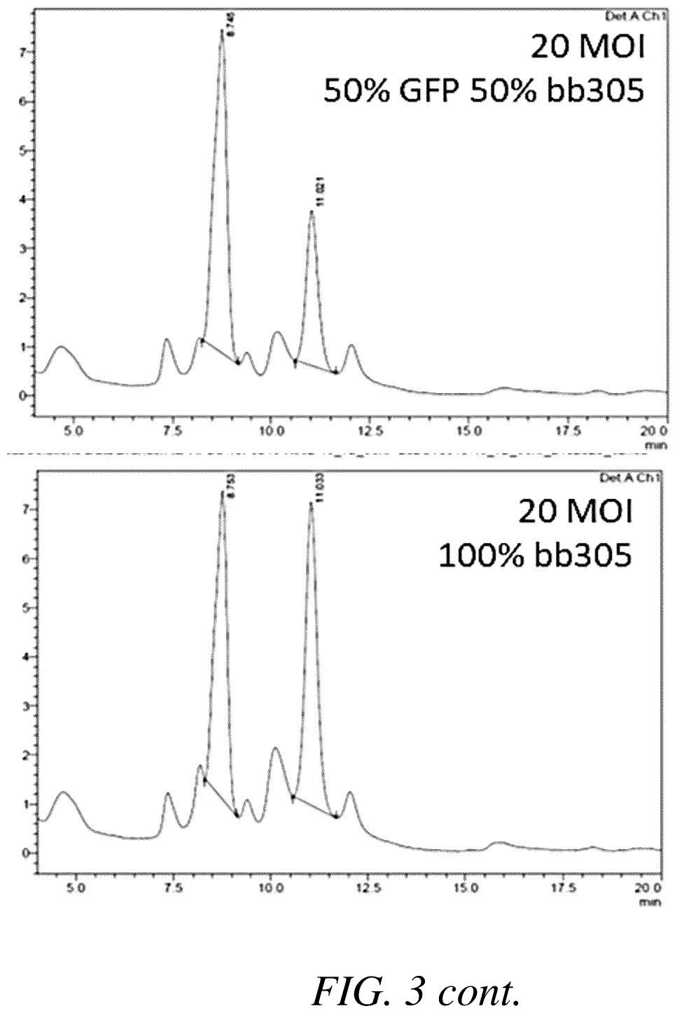

Based on the fits in FIG. 2 (and supported by the post-hoc analyses in Tables 3 and 4), a further experiment was conducted to examine MOIs of 10, 20, and 40 at harvest times of 48, 72, and 96 hours. The specificity of the method for detecting LVV was also assessed in this experiment. One lot of LVV test article (Lot 3, 1.82×10⁸ TU/mL) was prepared at 100%, 50%, and 0% relative activity by mixing with an off-target GFP LVV vector (3.42×10⁸ TU/mL). MOIs of 10, 20, and 40 were targeted in T-25 flasks based on the total transducing unit count (i.e., LentiGlobin BB305 LVV+GFP LVV). The test article consisting of 100% Lenti-GFP showed no analyte signal in the assay (FIG. 3), demonstrating the specificity of the method for detecting transduction-dependent HbA.

A preliminary assessment of accuracy was made by plotting the 100% LVV sample responses against the logarithm of the MOI (log₁₀) and fitting a linear regression. The responses from the 50% LentiGlobin BB305 LVV/50% Lenti-GFP sample were used to back-calculate MOI based on the curve fit. The back-calculated MOI was compared to the nominal MOI to determine the accuracy (Table 5). The 50% LentiGlobin BB305 LVV/50% Lenti-GFP sample demonstrated good accuracy and ranged from 44-54% at each harvest time point (with the exception of 67% at MOI 10 at 48 hours).

TABLE 5
Accuracy of 50% LentiGlobin BB305 LVV/50%
Lenti-GFP mixture in the assay

| | 48 h | 72 h | 96 h |
|---|---|---|---|
| MOI 10 | 67% | 43% | 47% |
| MOI 20 | 49% | 46% | 50% |
| MOI 40 | 45% | 42% | 44% |
| Average | 54% | 44% | 47% |

### Example 4

### Impact of Serum Lot and Cell Passage Number on HBF Expression

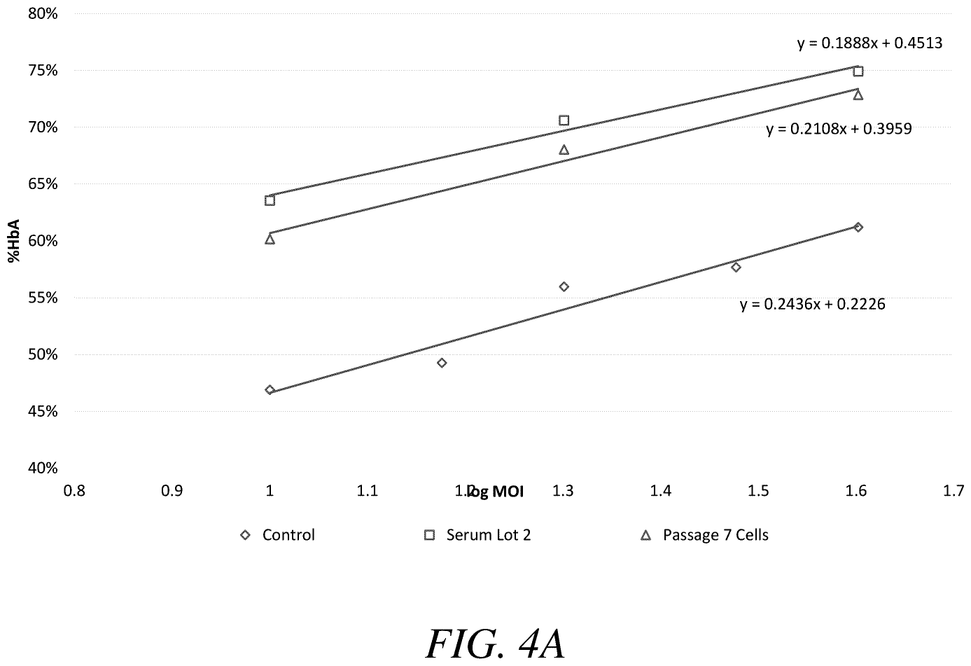

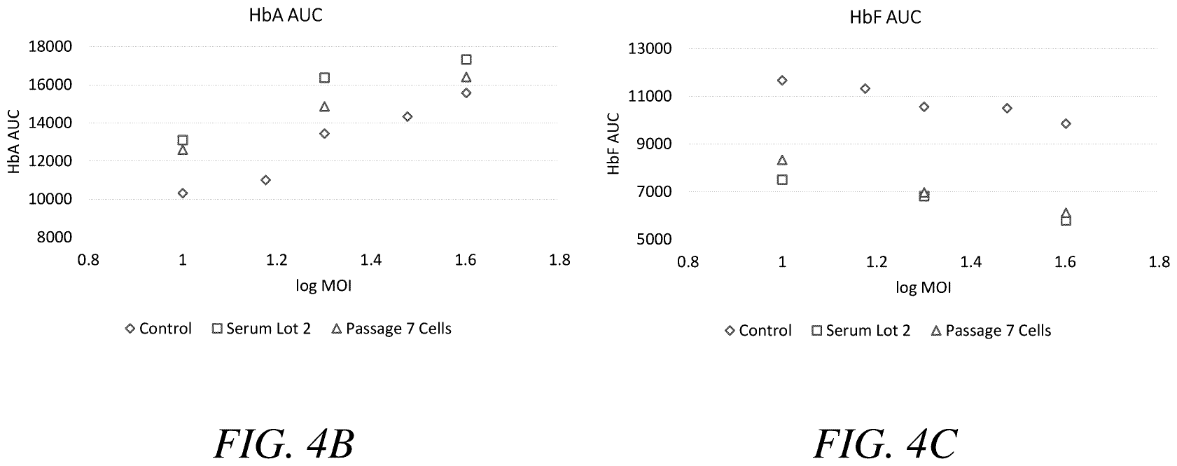

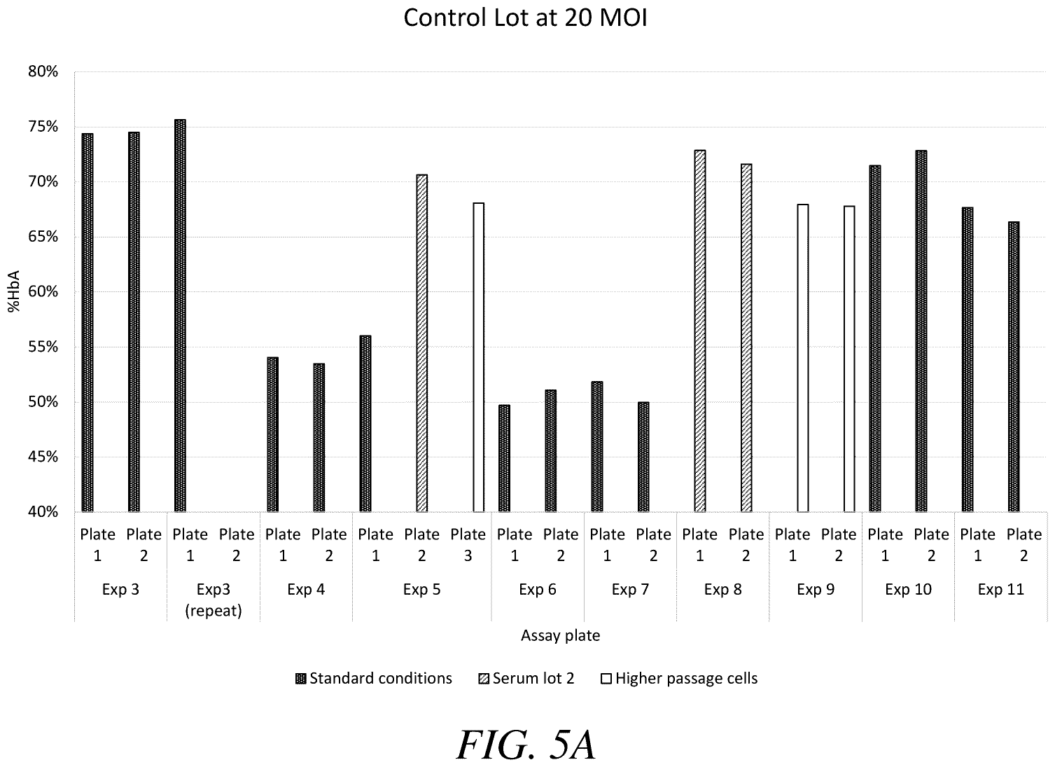

An experiment was designed to assess the impact of serum lot and cell passage number on the % HbA readout. Here, the standard cell culture conditions were tested against a different/second lot of FBS and against cells at a higher passage (versus the typical practice of using four days after thaw). Cells were thawed and cultured separately in two different lots of FBS and after 4 days 1×10⁶ cells were plated into a 24-well plate along with a third active cell culture maintained for a total of seven passages. Cells were transduced with adherent LVV at MOIs ranging from 10-40. The media was changed after approximately 24 hours and cells were harvested for Shimadzu HPLC analysis approximately 72 hours post-transduction. The results are presented in FIGS. 4A and 4B.

In this experiment, the second lot of serum and the later passage cells demonstrated markedly higher absolute % HbA values than the control condition (FIG. 4A). When dissecting out the HbA and HbF peak areas, the difference in peak areas is driven both by increased HbA and by decreased HbF (FIGS. 4B and 4C). Without wishing to be bound by any particular theory, these results suggest that the regulated expression of HbF in the K562 cells is impacted by the cell culture conditions, and this has an outsized impact on the absolute % HbA result.

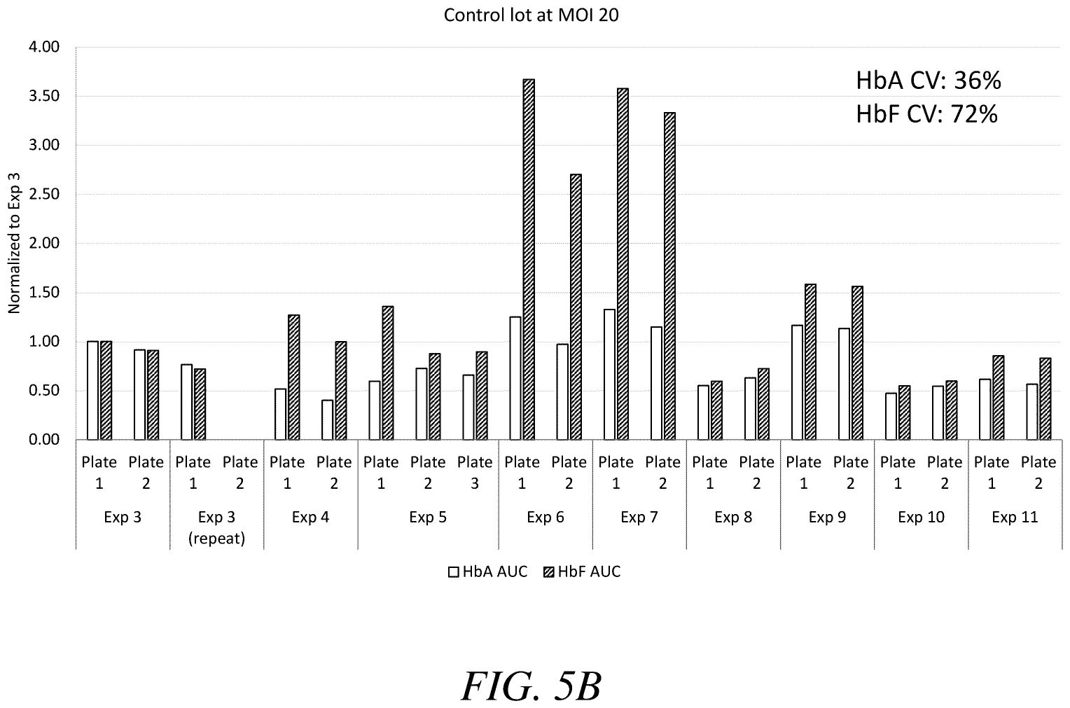

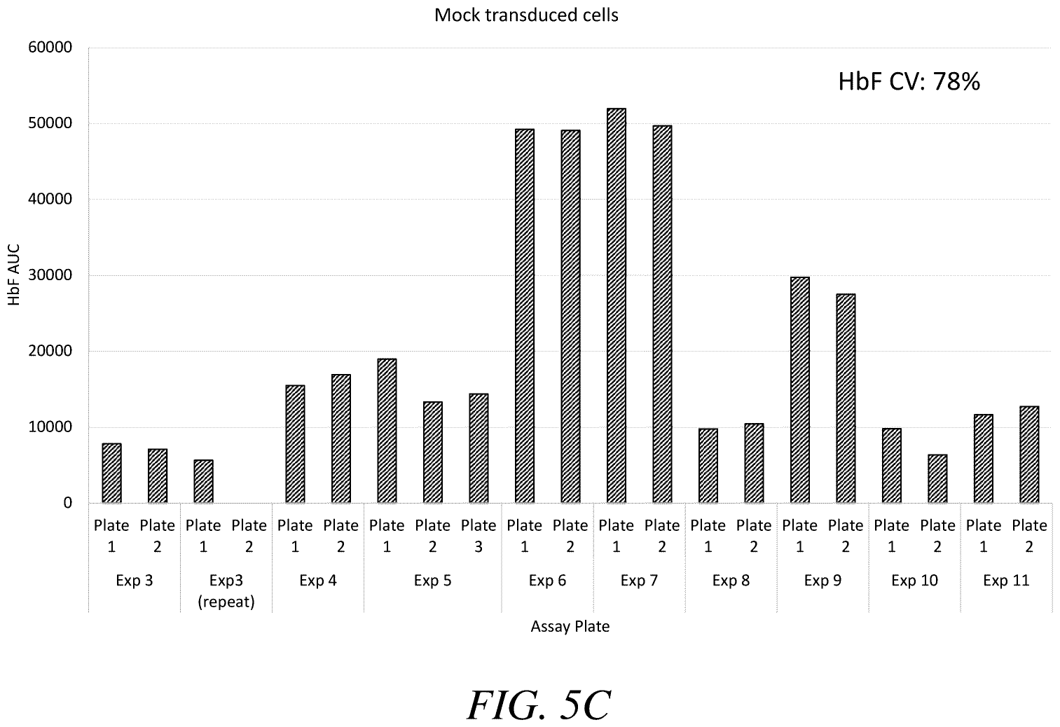

Another experiment was conducted to assess variability, and with the same methods describe above in this Example (see FIGS. 5A-5C). Variability of the % HbA is illustrated in FIG. 5A, which shows the % HbA results of the control LVV (lot 4) at MOI 20 over the course of testing. These data demonstrate additional variability indicative of unidentified factors that impact the absolute % HbA readout.

Moreover, FIG. 5B shows the relative variability of the HbA and HbF peak areas, which demonstrated CV values of 36% and 72%, respectively, across the assay plates tested. The HbA peak area variability captures the assay specific variation, indicating that the greater variability of the HbF peak area is due to factors extending beyond those related to the technical aspects of the method and are likely biological in nature (e.g., the regulated expression of endogenous HbF levels in K562 cells or the variation of cells in a given pellet).

Finally, FIG. 5C shows the HbF peak areas of the mock-transduced samples in each assay plate to show the peak area variability in isolation from the impact of LVV transduction. A similar pattern and variability of peak area (CV 78%) was observed.

### Example 5

### Relative Potency Calculation

Given the biological variability of HbF expression in K562 cells, a relative potency reportable result format was assessed as a way to measure potency of a given lot of lentiviral vector. In this regard, both parallel line and interpolation approaches were assessed and determined to accurately and precisely calculate a potency based on a reference standard.

The parallel line approach relies on fitting a dose response curve to a reference standard and a test article and using the fit parameters to determine the relative potency. In this approach an F-test, equivalence test, or other means of confirming parallel dose responses can be used, followed by fitting the reference and test articles to a common slope. The calculation uses the linear fit parameters of the log-dose response curve to determine relative potency according to the formula:

$$Relative potency = antilog ( \frac{Test intercept - Reference Intercept}{Common Slope} )$$

In an interpolation approach, a linear fit is applied to the reference standard log-dose response and the % HbA responses of the test article are used to interpolate MOI from the reference curve fit. The interpolated MOI relative to the nominal MOI is the relative potency (RP).

To test these approaches, a sufficient body of data was generated to be able to assess accuracy and intermediate precision by testing known dilutions of the reference vector and a second vector lot over multiple assay occasions (see Table 6). Specifically, K562 cells at a density of 1×10⁶ cells/mL were transduced in the presence of polybrene at MOIs of 10, 15, 20, 25, and 30 for the reference standard, 100% level, and test articles, at MOI 5, 7.5, 10, 12.5, and 15 for 50% level, and MOI 15, 22.5, 30, 37.5, and 45 for 150% level. The percent relative potency was calculated by the interpolation approach and the PLA approach and relative bias was determined using the formula: 100×((measured potency/target potency)−1). Test articles were assessed over multiple independent assay occasions and the average relative potency, standard deviation, and percent coefficient of variation was determined using the interpolation and PLA calculation approaches. In both instances the method demonstrated good precision with % CV less than or equal to 21%.

TABLE 6
Interpolation and Parallel line approach comparison

| Parameter | Desired target | Interpolation results | | PLA results | |
|---|---|---|---|---|---|
| Relative | Percent relative | 50% level: | 4% | 50% level: | 4% |
| Accuracy | bias ±30% | 100%: level | 23% | 100%: level | 10% |
| | | 100% level | 4% | 100% level | 1% |
| | | 150% level | −15% | 150% level | −17% |
| Intermediate | % CV ≤30% | Reference | Lot 5 | Reference | Lot 5 |
| Precision | | Test | Lot 6 | Test | Lot 6 |
| | | n | 3 | n | 3 |
| | | Average | 51% | Average | 49% |
| | | Std. Dev. | 11% | Std. Dev. | 10% |
| | | % CV | 21% | % CV | 21% |
| | | Reference | Reference | Reference | Reference |
| | | | Lot 19 | | Lot 19 |
| | | Test | Lot 4 | Test | Lot 4 |
| | | n | 3 | n | 3 |
| | | Average | 152% | Average | 143% |
| | | Std. Dev. | 19% | Std. Dev. | 13% |
| | | % CV | 13% | % CV | 9% |

### Example 6

### Method Sensitivity to Forced Degradation of LVV

Evaluations of stressed LVV material were performed at different points in the method development process and the results are presented in Table 7. The LVV was stressed by exposing to freeze thaw cycling conditions. Vector was removed from the freezer and exposed to ambient temperature for 3-6 hours and returning to ≤−65° C. for a minimum of 12 hours. A total of 3 and 5 freeze thaw cycles was performed for 2 vector lots. The vector was applied to K562 cells plated in a 24-well plate at 1×10⁶ cells/mL at target MOIs ranging between 10-40 (based on pre-stress titer). Lysates were analyzed using Waters UHPLC equipped with a TUV detector, and Poly LC PolyCAT A 150×1.0 mm, 5 μm, 1000 A column. The potency relative to the unstressed material was calculated based on the interpolation approach.

In two preparations of lot 4 and one preparation of lot 1, stressing by three repeated freeze thaws resulted in relative potencies (RP) of 37-62% relative to the unstressed lot, and stressing by five repeated freeze thaws resulted in potencies of less than 25% to 49% relative to the unstressed lot. Patient derived CD34+ cells were also transduced with this stressed material and tested for vector copy number (VCN) and a functional assay measuring cell enucleation and a comparable reduction in response was observed, supporting the capacity of the method to detect reductions in LVV functional activity (data not shown).

Furthermore, in a presumed second mechanism of stress induced reduction in activity, lot 6 left on the benchtop for two days demonstrated a potency of less than 25% relative to the unstressed lot.

TABLE 7
Analysis of freeze-thaw and benchtop stressed LVV

| | | | Result |
|---|---|---|---|
| Lot | Degradation | LC method | (% RP) ¹ |
| Lot 4 (prep 1) | 3x F/T | Shimadzu | 62% |
| | 5x F/T | Shimadzu | 49% |
| Lot 4 (prep 2) | 3x F/T | Waters TUV | 41% |
| | 5x F/T | Waters TUV | <25% (16%)² |
| Lot 1 | 3x F/T | Waters TUV | 37% |
| | 5x F/T | Waters TUV | <33% (28%)² |
| Lot 6 | 2d benchtop | Waters TUV | <25% (15%)² |

¹ Results are relative to lot-matched unstressed material.
²Values in parentheses are extrapolated beyond the range of the standard curve and are included for information.

### Example 7

### Method Repeatability

Repeatability of the UPLC assay was assessed by injecting three groups of three technical replicates of AFSC (hemoglobin control standard containing a mix of HbA, HbF, HbS, and HbC), LVV transduced K562, and mock transduced K562 over the course of a sequence of over 52 injections. Lysates were prepared by resuspending the cell pellet in 100 μL of Milli-Q water and vortexed for 15 seconds. Lysates were spun down and supernatants injected for UPLC analysis to determine the consistency of retention time and peak area of these over the course of the sample sequence.

Table 8 shows that the CV across all the replicates for retention time and peak area of the HbA and HbF peaks was ≤2%, indicating good repeatability. Furthermore, the change in retention time and peak area between the first and last injection within each group series was less than ±5% different from the average suggesting within run consistency over the duration of >50 injections.

TABLE 8
Assay repeatability results

| AFSC | | | | | Transduced cells | | | | | Mock-transduced cells | | |
|---|---|---|---|---|---|---|---|---|---|---|---|---|
| | HbA | | HbF | | | HbA | | HbF | | | HbF | |
| | Retention | Peak | Retention | Peak | | Retention | Peak | Retention | Peak | | Retention | Peak |
| #¹ | time | AUC | time | AUC | #¹ | time | AUC | time | AUC | #¹ | time | AUC |
| 1 | 3.378 | 17721 | 2.405 | 19647 | 5 | 3.422 | 21902 | 2.377 | 6322 | 3 | 2.380 | 9631 |
| 4 | 3.380 | 17953 | 2.405 | 19487 | 7 | 3.420 | 21276 | 2.378 | 6190 | 6 | 2.379 | 9755 |
| 13 | 3.377 | 18188 | 2.404 | 19863 | 14 | 3.427 | 21566 | 2.377 | 6358 | 12 | 2.375 | 9459 |
| 20 | 3.377 | 17999 | 2.406 | 19678 | 21 | 3.420 | 20899 | 2.378 | 6136 | 19 | 2.380 | 9330 |
| 27 | 3.381 | 17757 | 2.407 | 20107 | 28 | 3.422 | 20447 | 2.382 | 5981 | 26 | 2.382 | 9393 |
| 34 | 3.377 | 18045 | 2.406 | 20031 | 35 | 3.428 | 21491 | 2.390 | 6104 | 33 | 2.381 | 9264 |
| 41 | 3.378 | 17923 | 2.406 | 19937 | 42 | 3.426 | 21230 | 2.379 | 6215 | 40 | 2.383 | 9385 |
| 48 | 3.376 | 18027 | 2.407 | 19947 | 49 | 3.426 | 21365 | 2.381 | 6041 | 47 | 2.377 | 9321 |
| 51 | 3.378 | 18124 | 2.407 | 20007 | 52 | 3.426 | 20953 | 2.385 | 6165 | 50 | 2.382 | 9306 |
| Avg | 3.378 | 17971 | 2.406 | 19856 | Avg | 3.424 | 21237 | 2.381 | 6168 | Avg | 2.380 | 9427 |
| CV | 0% | 1% | 0% | 1% | CV | 0% | 2% | 0% | 2% | CV | 0% | 2% |
| Δ² | 0.000 | 403 | 0.002 | 360 | Δ² | 0.004 | −949 | 0.008 | −157 | Δ² | 0.002 | −325 |

¹Injection number
²Δ last-first injection

### Example 8

### Lentiviral Lot Testing

To examine the reproducibility of the potency assay, several lentiviral lots were tested under the same assay conditions. In brief, 1×10⁶ K562 cells were seeded in each well of a 24-well plate and transduced with LVV at MOIs 0, 10, 15, 20, 25, and 30 in duplicate. Cell transduction was performed in the presence of 8 μg polybrene. After transduction, the cells were maintained for three days (approximately 72 hours). Cells were then harvested and analyzed by UHPLC per example 10 with the exception that columns were conditioned with K562 lysate and a TUV detector was used. Percent relative potency was calculated using the interpolation approach. A summary of the lot testing results is presented in Table 9, with each of the test plates passing internal acceptance criteria.

TABLE 9
Summary results of LVV lot testing relative to reference lot 19

| | | CV of | Control | | |
|---|---|---|---|---|---|
| Test article/ | R² | replicates | (70- | Mock | |
| LVV lot | (>0.9) | (≤20%) | 130%) | (≤5%) | % RP |
| Lot 4 (prep 1) | 1.0 | <5% | N/A | Undetected | 140% |
| Lot 4 (prep 2) | 1.0 | <5% | N/A | Undetected | 166% |
| Lot 4 (prep 3) | 1.0 | <5% | 112% | Undetected | 147% |
| Lot 1 | 1.0 | <10% | 107% | Undetected | 48% |
| Lot 2 | 1.0 | <10% | 101% | Undetected | 100% |
| Reference | 1.0 | <5% | 96% | ≤5% | 51% |
| Lot 19 - 50% | | | | | |
| Reference | 1.0 | <10% | 105% | ≤5% | 126% |
| Lot 19 - 150% | | | | | |
| Suspension Lot 003 | 1.0 | ≤5% | 96% | ≤5% | 143% |
| Suspension Lot 004 | 1.0 | ≤5% | 105% | ≤5% | 142% |
| Lot 6 | 1.0 | ≤5% | 116% | Undetected | 74% |
| Lot 7 (Current FBS) | 0.9 | ≤5% | 111% | Undetected | 91% |
| Lot 7 (Future FBS) | 1.0 | ≤5% | 105% | Undetected | 93% |
| Reference | 1.0 | ≤5% | 104% | Undetected | 118% |
| Lot 19 - 100% | | | | | |
| (Current FBS) | | | | | |
| Reference | 0.9 | ≤5% | 112% | Undetected | 105% |
| Lot 19 - 100% | | | | | |
| (Future FBS) | | | | | |

The data demonstrate reproducible performance of the method and a range of relative potency responses above and below the selected LVV reference lot. Repeatable tests of the same lot demonstrate acceptable method precision and targeted dilutions of the reference lot demonstrate acceptable method accuracy.

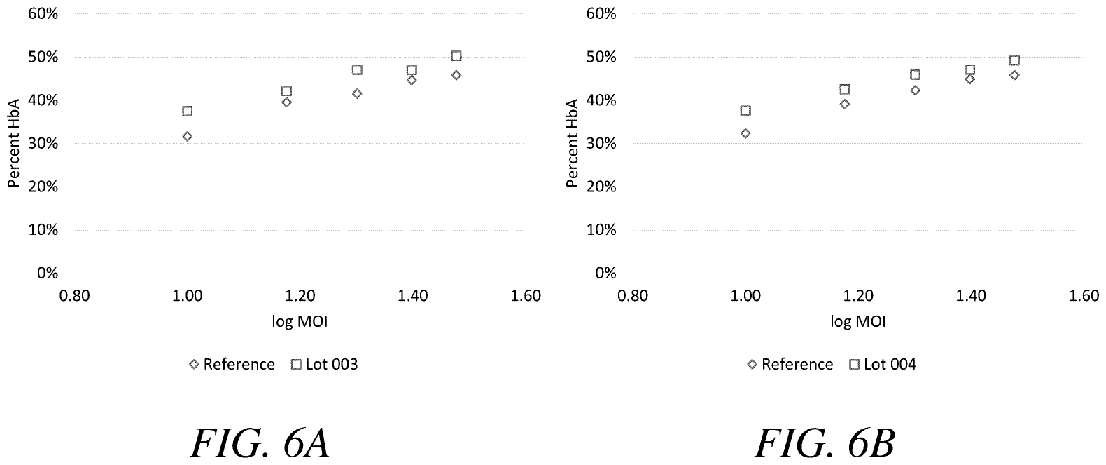

The suspension LVV lots 003 and 004 exhibited dose-response curves similar to the adherent LVV reference lot 19 (FIGS. 6A and 6B). This suggests the method may be suitable for use with suspension LVV products with minimal or no adaptation.

### Example 9

### Method Linearity

Linearity was assessed by diluting a transduced K562 lysate into mock transduced K562 lysate. The mock transduced lysate contains HbF, but not HbA, thus allowing for the assessment of changes in percent HbA as the component ratios change.

In brief, the diluted lysates were analyzed via waters UHPLC equipped with TUV detector at levels ranging from 100% transduced to 0% transduced lysate. Peak areas were determined for each level and in total those results were used to create a linear curve. From this curve biases of each level were determined.

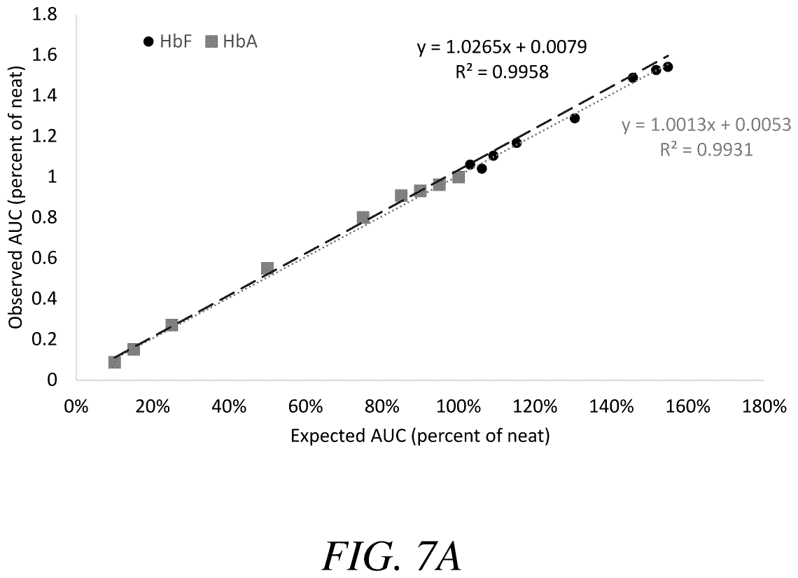

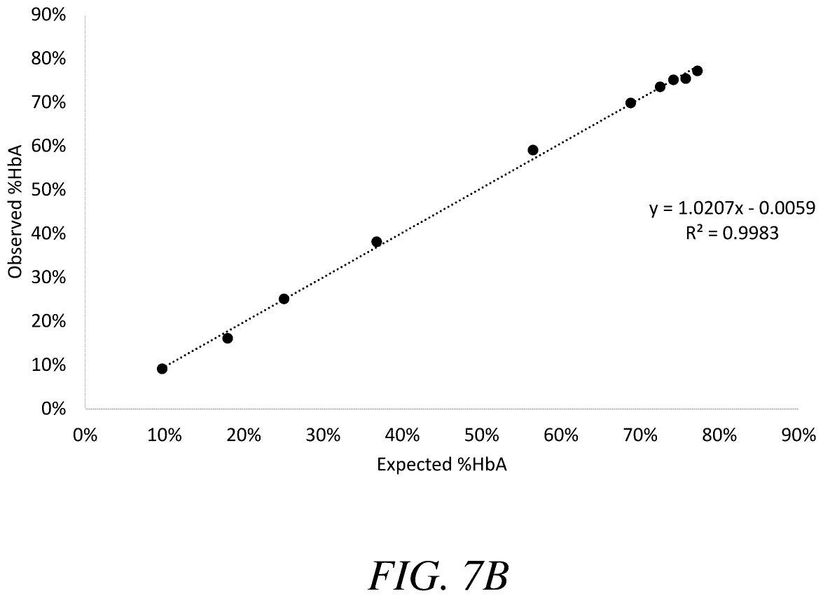

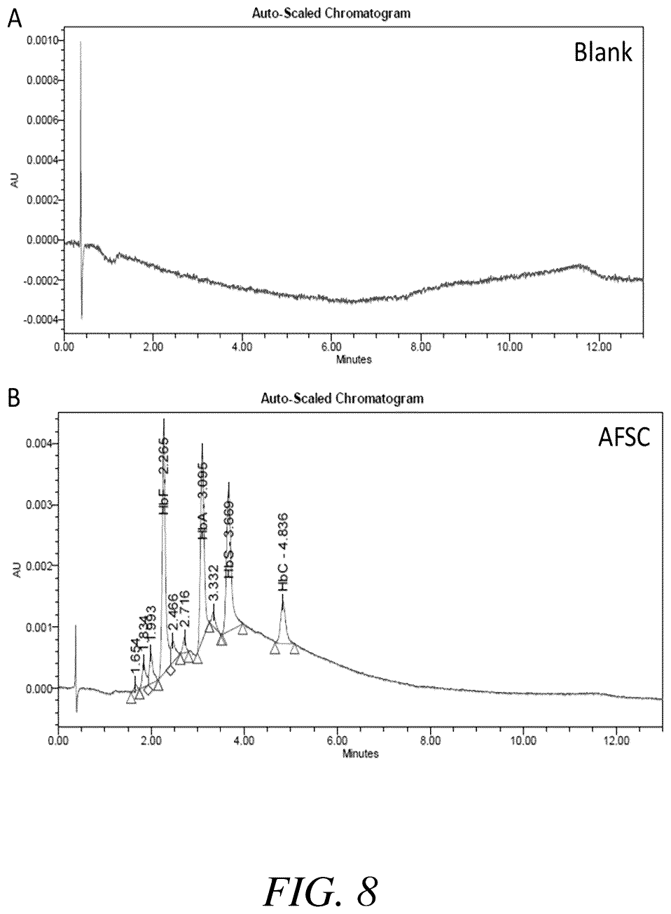

The data are presented in Table 10. A linear relationship in peak AUC is observed when transduced lysate is diluted down to 15% of the neat concentration, as evidenced by the percent relative bias at each dilution level falling within ±10%. In addition, the plot of expected vs observed peak AUC demonstrates a slope of 1.00 and 1.03 indicating overall linearity for the HbF and HbA peaks, respectively (FIG. 7A). Retention time was unchanged for each analyte, with the maximum difference in peak retention time across the dilution series falling <0.02 minutes (data not shown). As the transduced lysate is diluted with a mock transduced lysate expressing HbF the total % HbA is expected to decrease as the HbA analyte is diluted down while the HbF analyte remains relatively constant. When the expected % HbA is calculated and plotted against the observed % HbA a slope of 1.02 is observed (FIG. 7B) with the percent relative bias within ±10% at each point (data not shown).

Taken together, the method demonstrates linearity of a transduced lysate down to 5% of the neat transduced levels of % HbA, which corresponds to approximately 10% HbA. This lower range of linearity is below the lowest % HbA levels observed in stressed LVV samples demonstrating greater than 50% loss in potency.

TABLE 10
Linearity data showing observed vs. expected and relative bias.

| Percent of | HbF | | | | HbA | | | | % HbA | | |
|---|---|---|---|---|---|---|---|---|---|---|---|
| Transduced | Observed | | Expected | Relative | Observed | | Expected | Relative | | | Relative |
| Material | AUC | % CV | AUC | Bias | AUC | % CV | AUC | Bias | Expected | Observed | Bias |
| 100% | 5941 | 2% | 5941 | N/A | 20073 | 0.8% | 20073 | N/A | 77% | 77% | 0% |
| 95% | 6302 | 4.1% | 6121 | 3% | 19307 | 2.4% | 19069 | 1% | 76% | 75% | 0% |
| 90% | 6186 | 1.4% | 6302 | −2% | 18699 | 2.3% | 18065 | 4% | 74% | 75% | 1% |
| 85% | 6565 | 4.0% | 6483 | 1% | 18248 | 4.3% | 17062 | 7% | 72% | 74% | 1% |
| 75% | 6940 | 2.5% | 6844 | 1% | 16083 | 7.1% | 15054 | 7% | 69% | 70% | 2% |
| 50% | 7660 | 3.9% | 7748 | −1% | 11065 | 5.3% | 10036 | 10% | 56% | 59% | 5% |
| 25% | 8842 | 2.4% | 8651 | 2% | 5452 | 9.4% | 5018 | 9% | 37% | 38% | 4% |
| 15% | 9068 | 3.3% | 9013 | 1% | 3043 | 7.2% | 3011 | 1% | 25% | 25% | 0% |
| 10% | 9160 | 3.5% | 9193 | 0% | 1760 | 10.0% | 2007 | −12% | 18% | 16% | −10% |
| 5% | 9273 | 2.1% | 9374 | −1% | 932 | 5.1% | 1004 | −7% | 10% | 9% | −6% |
| 0% | 9555 | 3.9% | N/A | N/A | N/A | N/A | N/A | N/A | N/A | N/A | N/A |

### Example 10

### Illustrative Ion-Exchange UPLC Method

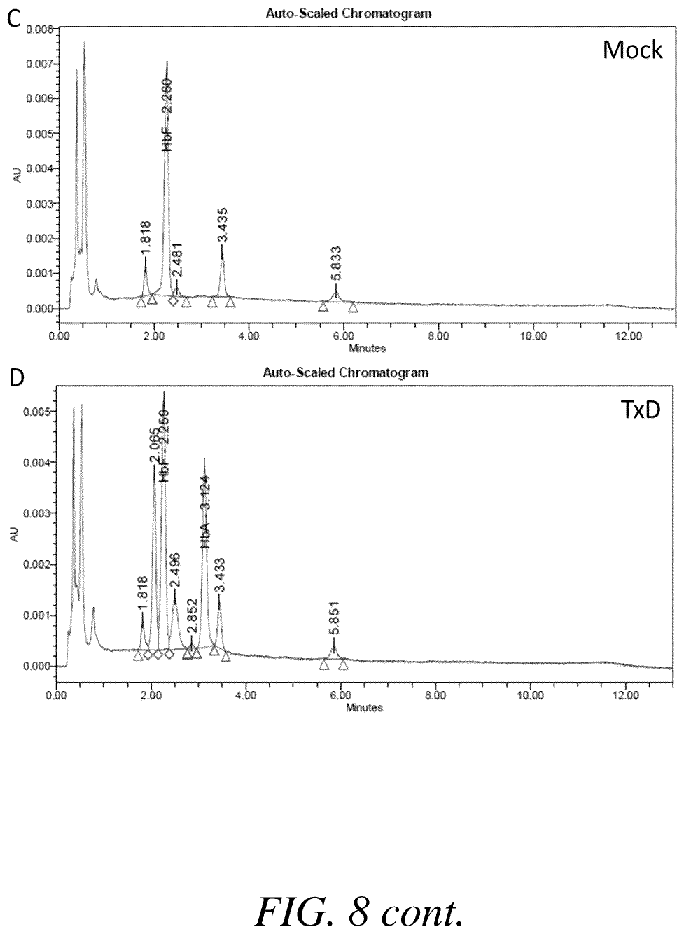

K562 cells seeded at a density of 1×10⁶ cells/mL were transduced with LVV in the presence of polybrene at MOI 20. The media was changed after 24 hours in culture and the cells were harvested and frozen down as cell pellets 72 hours after transduction. Transduced K562 cell pellets were prepared by lysing in 100 μL of Milli-Q water, vortexed, clarified by centrifugation, and injected into a Waters ACQUITY I-class system with a PolyLC PolyCAT A 150×1.0 mm, 5 μM, 1000 Angstrom, column conditioned with Triethylamine (TEA), and measured using a PDA detector. First and second mobile phases were tris-buffered KCN with and without 0.2M NaCl, respectively, pH 6.5. Results are shown in FIG. 8, which demonstrate sufficient peak separation and identification. Together, the inventors have surprisingly discovered methods for assessing HbA expression and vector potency that demonstrates specificity, robustness, precision, and linearity across a range that is suitable for the determination of % HbA (as calculated from the constituent HbA and HbF analyte peaks) in cell pellet samples produced from the hemoglobin formation bioassay.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

What is claimed is:

1. A method for assessing hemoglobin A (HbA) formation in cells comprising:
a) modifying a population of cells to express β-globin;
b) lysing the cells under non-denaturing conditions, thus forming cell lysates;
c) analyzing the cell lysates with ion exchange (IEX) chromatography comprising:
i) passing the cell lysates through an IEX chromatographic column;
ii) detecting heme groups associated with HbF and/or HbA hemoglobin multimers at 418 nm;
d) calculating HbA expression.

2. The method of claim 1, wherein the modifying comprises introducing a vector encoding a β-globin gene into the population of cells.

3. The method of claim 2, wherein the vector is a viral vector or a non-viral vector.

4. The method of any one of the preceding claims, wherein the vector is introduced by transfection, transduction, or electroporation.

5. The method of claim 1, wherein the modifying comprises introducing into the population of cells:
a) an endonuclease or polynucleotide encoding an endonuclease;
b) and a donor repair template encoding a β-globin.

6. The method of claim 5 wherein the endonuclease is selected from the group consisting of:
a) a homing endonuclease, or functional variant thereof;
b) a megaTAL, or functional variant thereof;
c) a CRISPR-associated nuclease, or functional variant thereof;
d) a zinc-finger nuclease, or functional variant thereof; and
e) transcription activator-like effector nuclease (TALEN), or functional variant thereof.

7. The method of claim 5 or claim 6, wherein the endonuclease or polynucleotide encoding an endonuclease is introduced by transfection, transduction, or electroporation.

8. The method of any one of claims 5-7, wherein the donor repair template is introduced by transfection, transduction, or electroporation.

9. The method of any one of the preceding claims, wherein the method further comprises culturing the cells for about 24 to about 96 hours post-modifying.

10. A method for assessing potency of a viral vector encoding a β-globin gene comprising:
a) transducing a population of cells that do not express hemoglobin A (HbA) with a vector encoding a β-globin gene;
b) lysing the cells under non-denaturing conditions, thus forming cell lysates;
c) analyzing the cell lysates with ion exchange (IEX) chromatography comprising:
i) passing the cell lysates through an IEX chromatographic column;
ii) detecting heme groups associated with HbF and/or HbA hemoglobin multimers at 418 nm;
d) calculating HbA expression relative to HbA expression in a cell introduced with a reference standard vector.

11. The method of claim 4, wherein the potency is a relative potency.

12. The method of claim 10 or 11, wherein the method further comprises culturing the population of cells for 24 to 96 hours post-transduction.

13. The method of any one of the preceding claims, wherein the population of cells do not endogenously express HbA.

14. The method of any one of the preceding claims, wherein the population of cells have been genetically edited to not express HbA.

15. The method of any one of the preceding claims, wherein the population of cells express fetal hemoglobin (HbF).

16. The method of any one of the preceding claims, wherein the population of cells are a myelogenous leukemia cell line.

17. The method of any one of the preceding claims, wherein the population of cells are K562 cells.

18. The method of any one of the preceding claims, wherein the cells are plated at a cell density of about 0.5×10⁶ cells/ml, about 1.0×10⁶ cells/ml, about 1.5×10⁶ cells/ml, about 2.0×10⁶ cells/ml, about 2.5×10⁶ cells/ml, or about 3.0×10⁶ cells/ml prior to modification or transduction.

19. The method of any one of claims 1-18, wherein the cells are plated at a cell density of about 0.5×10⁶ cells/ml to about 3.0×10⁶ cells/ml prior to modification or transduction.

20. The method of any one of claims 1-18, wherein the cells are plated at a cell density of about 0.5×10⁶ cells/ml to about 2.5×10⁶ cells/ml prior to modification or transduction.

21. The method of any one of claims 1-18, wherein the cells are plated at a cell density of about 0.5×10⁶ cells/ml to about 2.0×10⁶ cells/ml prior to modification or transduction.

22. The method of any one of claims 1-18, wherein the cells are plated at a cell density of about 0.5×10⁶ cells/ml to about 1.5×10⁶ cells/ml prior to modification or transduction.

23. The method of any one of claims 1-18, wherein the cells are plated at a cell density of about 0.6×10⁶ cells/ml to about 1.4×10⁶ cells/ml prior to modification or transduction.

24. The method of any one of claims 1-18, wherein the cells are plated at a cell density of about 0.7×10⁶ cells/ml to about 1.3×10⁶ cells/ml prior to modification or transduction.

25. The method of any one of claims 1-18, wherein the cells are plated at a cell density of about 0.8×10⁶ cells/ml to about 1.2×10⁶ cells/ml prior to modification or transduction.

26. The method of any one of claims 1-18, wherein the cells are plated at a cell density of about 0.9×10⁶ cells/ml to about 1.1×10⁶ cells/ml prior to modification or transduction.

27. The method of any one of claims 1-18, wherein the cells are plated at a cell density of about 1.0×10⁶ cells/ml prior to modification or transduction.

28. The method of any one of claims 1-27, wherein the cells are plated in tissue culture flasks.

29. The method of any one of claims 1-27, wherein the cells are plated in a 12-well plate.

30. The method of any one of claims 1-27, wherein the cells are plated in a 24-well plate.

31. The method of claim 29 or claim 30, wherein the cells are plated in a total volume of about 1 ml.

32. The method of claim 29 or claim 30, wherein the cells are plated in a total volume of about 2 ml.

33. The method of any one of claims 1-32, wherein the cells are transduced in the presence of polybrene.

34. The method of claim 33, wherein the cells are transduced in the presence of about 2 μg/ml to about 8 μg/ml polybrene.

35. The method of claim 33, wherein the cells are transduced in the presence of about 8 μg/ml polybrene.

36. The method of any one of claims 1-35, wherein the cells are cultured for about 48 to about 96 hours post-modification or -transduction.

37. The method of any one of claims 1-35, wherein the cells are cultured for about 60 to about 84 hours post-modification or -transduction.

38. The method of any one of claims 1-35, wherein the cells are cultured for about 48, about 60, about 72, about 84, or about 96 hours post-modification or -transduction.

39. The method of any one of claims 1-35, wherein the cells are cultured for about 72±2 hours post-modification or -transduction.

40. The method of any one of claims 1-35, wherein the cells are cultured for about 72 hours post-modification or -transduction.

41. The method of any one of claims 1-40, wherein the cells are frozen after lysis and prior to analyzing the cell lysates with ion exchange (IEX) chromatography.

42. The method of any one of claims 1-41, wherein the HbA comprises α and β globin chain dimers or tetramers.

43. The method of any one of claims 7-42, wherein the HbF comprises α and γ globin chain dimers or tetramers.

44. The method of any one of claims 1-43, wherein the β-globin is a human β-globin.

45. The method of any one of claims 1-44, wherein the β-globin is β^{A-T87Q} globin, a β^{A-G16D/E22A/T87Q}-globin, or a β^{A-T87Q/K95E/K120E}-globin.

46. The method of any one of claims 1-45, wherein the vector is a lentiviral vector.

47. The method of any one of claims 1-46, wherein the vector is an AnkT9W vector, a T9Ank2W vector, a TNS9 vector, a lentiglobin HPV569 vector, a lentiglobin BB305 vector, a BG-1 vector, a BGM-1 vector, a d432βAγ vector, a mLARβΔγV5 vector, a GLOBE vector, a G-GLOBE vector, a βAS3-FB vector, a V5 vector, a V5m3 vector, a V5m3-400 vector, and a G9 vector, or a derivative thereof.

48. The method of claim 47, wherein the vector is bb305.

49. The method of any one of claims 1-48, wherein the transducing comprises transduction of vector at a multiplicity of infection (MOI) of about 5 to about 40, about 5 to about 30, about 10 to about 40, or about 10 to about 30.

50. The method of any one of claims 1-48, wherein the transducing comprises transduction of vector at a multiplicity of infection (MOI) of 5-40, 5-30, 10-40, or 10-30.

51. The method of any one of claims 1-48, wherein the transducing comprises transduction of vector at a multiplicity of infection (MOI) of about 5, about 10, about 15, about 20, about 25, about 30, about 35, and/or about 40.

52. The method of any one of claims 1-48, wherein the transducing comprises transduction with vector at a multiplicity of infection (MOI) of about 20.

53. The method of any one of claims 1-52, wherein the transducing comprises transduction with vector at one or more MOIs in different wells or plates.

54. The method of any one of claims 1-53, wherein the transducing comprises transduction with vector at one or more MOIs in different wells or plates in duplicate.

55. The method of any one of claims 1-54, wherein the transducing comprises transduction with vector at one or more MOIs in different wells or plates in triplicate.

56. The method of any one of claims 1-55, wherein the transducing comprises transduction with vector at MOIs of 10, 15, 20, 25, and 30.

57. The method of any one of claims 1-56, wherein the IEX chromatography is IEX HPLC.

58. The method of any one of claims 1-56, wherein the IEX chromatography is IEX UPLC.

59. The method of any one of claims 1-56, wherein the IEX chromatography is IEX UHLPC.

60. The method of any one of claims 1-59, wherein the IEX chromatography comprises liquid-based first and second mobile phases.

61. The method of any one of claims 1-60, wherein the column comprises a solid phase comprising aspartic acid chains covalently linked to a substrate.

62. The method of any one of claims 1-61, wherein the column comprises a solid phase comprising sulfonic acid ligands covalently lined to a substrate.

63. The method of any one of claims 1-62, wherein the substrate is a silica substrate.

64. The method of any one of claims 1-62, wherein the substrate is a polymer.

65. The method of any one of claims 1-64, wherein the chromatography comprises a tunable ultraviolet (TUV) detector.

66. The method of any one of claims 1-64, wherein the chromatography comprises a photodiode array ultraviolet (PDA UV) detector.

67. The method of any one of claim 1-66, wherein the chromatography separates HbF multimers from HbA multimers.

68. The method of any one of claims 1-67, wherein chromatographic identification of HbA and HbF is made based on the matched retention time of the analyte peaks relative to a hemoglobin standard.

69. The method of claim 68, wherein the standard is AFSC.

70. The method of any one of claims 1-69, wherein the calculating comprises determining an HbA peak and measuring the area under the curve (AUC).

71. The method of any one of claims 1-70, wherein the calculating comprises determining an HbF peak and measuring the area under the curve (AUC).

72. The method of any one of claims 1-71, wherein the calculating comprises determining HbA expression as a percentage of HbA relative to the sum of HbA and HbF.

73. The method of any one of claims 1-72, wherein the calculating further comprises fitting a log-dose response curve to the calculated HbA expression.

74. The method of any one of claims 1-73, wherein the calculating further comprises fitting a linear log-dose response curve to a reference standard and the vector.

75. The method of claim 73 or claim 74, wherein the log-dose is a log₁₀ dose.

76. The method of any one of claim 73-75, wherein the fitting comprises a parallel line approach to determine a relative potency.

77. The method of claim 76, wherein the relative potency is determined by the formula:
$$Relative potency = antilog ( \frac{Test intercept - Reference Intercept}{Common Slope} )$$

78. The method of any one of claims 73-75, wherein the fitting comprises an interpolation approach.

79. The method of claim 78, wherein the interpolation approach comprises a linear fit applied to the reference standard log-dose response and the % HbA responses of the vector are used to interpolate MOI from the reference curve fit.

80. The method of any one of the preceding claims, wherein the method is an in vitro method.

81. The method of any one of the preceding claims, wherein the method is an ex vivo method.
